(19) United States
(12) Patent Application Publication
(10) Pub. No.: US 2024/0133903 A1
(43) Pub. Date: Apr. 25, 2024

(54) System, Apparatus, and Method of Testing a Blood Sample or a Non-Blood Sample Capable of Transporting Oxygen in Order to Generate an Oxygen Equilibrium Curve

(71) Applicant: OxyDial Inc, Philadelphia, PA (US)

(72) Inventor: Alexey Aprelev, Philadelphia, PA (US)

(21) Appl. No.: 18/379,919

(22) Filed: Oct. 12, 2023

Related U.S. Application Data
(60) Provisional application No. 63/380,571, filed on Oct. 22, 2022.

(51) Int. Cl. G01N 33/80; G01N 21/31; G01N 21/64

(52) U.S. Cl. CPC G01N 33/80; G01N 21/31; G01N 21/6428; G01N 2021/6439

(57) ABSTRACT

System, apparatus and method of testing a sample capable of transporting oxygen and including a dye, wherein the system includes: an illumination device to generate a first light pulse for a first period and second light pulses for each second period, the first pulse and second pulses illuminating the sample; a first detector to collect absorbance spectrum measurements from the sample illuminated by the first pulse; a second detector to collect phosphorescence measurements emitted by the dye of the sample illuminated by the second pulses, each phosphorescence measurement collected during a third period after a corresponding second pulse; a microcontroller to generate an absorbance spectrum average of the spectrum measurements, and to generate a phosphorescence average of phosphorescence measurements; and a computing device to calculate SatHb based on the spectrum average and calculate PO2 based on the phosphorescence average, and plot a point SatHb, PO2 on an oxygen equilibrium curve.

### CROSS REFERENCE TO RELATED APPLICATIONS

This application claims benefit of U.S. Provisional Patent Application No. 63/380,571, filed on Oct. 22, 2022, the contents of which are incorporated herein by reference in their entirety.

### BACKGROUND

### Field

The present application relates to systems, methods, and apparatus directed to testing blood samples. More specifically, the present application is directed to a system, apparatus, and method of testing a blood sample or a non-blood sample capable of transporting oxygen in order to generate an oxygen equilibrium curve.

### Brief Discussion of Related Art

Gas saturation of a solute at different pressures of the gas in solution is important in characterizing multiple gas transfer processes in nature as well as technology. An especially important case involves blood that carries oxygen in living organisms. Oxygen transfer to the organs is possible as a result of blood flow from the lungs to the tissues. In particular, blood can be described as an emulsion of a hemoglobin solution, wherein droplets of concentrated hemoglobin (Hb) are suspended in plasma.

In order to transfer oxygen efficiently, blood has to be able to easily bind to oxygen in the lungs and readily free the oxygen in tissues of the living organisms. This is achieved by so-called allosteric effect. In particular, every molecule of hemoglobin binds up to four (4) molecules of oxygen, wherein each subsequent molecule of hemoglobin binds easier than the previous one to oxygen molecules, thus showing strong cooperativity or allosteric effect.

An oxygen association/dissociation curve can be used in order to graphically depict hemoglobin's unique ability to transfer oxygen to the tissues. In particular, the oxygen association-dissociation curve is an X-Y graph with an X-axis that represents an oxygen partial pressure in fluid and a Y-axis that represents a hemoglobin-oxygen saturation.

For generating such an oxygen association/dissociation curve, a blood sample of completely oxygenated blood is used at the start and a partial pressure of oxygen (PO2) and oxygen saturation of hemoglobin (SatHb) are periodically measured in the blood sample, while the oxygen is removed from the blood sample. When the aforementioned curve is generated slowly enough, it is called an Oxygen Equilibrium Curve (OEC).

As will be discussed below in greater detail, current methods of generating an OEC generally use electrochemical systems that present significant drawbacks. In particular, the electrodes that are used in the electrochemical analysis are notoriously not stable and need everyday calibration. This represents a major source of error in data collection of PO2 and SatHb from the blood sample. In the foregoing regard, the electrodes cannot be miniaturized and a large blood sample is required (i.e., several milliliters of blood), presenting serious and challenging drawbacks in the current method and systems.

The current systems and methods generally use three (3) basic approaches for deoxygenating the blood sample, while measuring PO2 and SatHb of the blood sample.

In particular, nitrogen bubbling through the blood sample is used in order to deoxygenate the blood sample. While this is a reliable traditional method, the method does have several drawbacks. First, a large amount of blood is required as a blood sample (i.e., several milliliters of blood). Second, the blood sample must be continuously mixed using a magnetic bar. As a result, the optical cuvette that contains the blood sample for analysis must be considerably large, and thus entails a problem of providing thermo-stabilization to the optical cuvette.

In measuring PO2, an electrochemical method called the Clark electrode is used. While this is a reliable traditional method (since 1953), the method does has several drawbacks. More specifically, the method requires a cumbersome calibration of the electrode and a long stabilization period. The Clark electrode requires everyday calibration and replacement of protective membranes, especially when the electrode is used for protein solutions.

In measuring SatHb, current instruments use a rather primitive two (2)-wavelength approach, having several drawbacks. This approach entails the use of a pair of photo sensors with band-pass filter, or a pulse oximeter that generally uses a similar approach. While this approach allows dramatically decreasing the cost because of its simplicity, it nonetheless lowers the quality of the measurements and works only for a known spectrum of hemoglobin (Hb) without any contamination. Moreover, instead of measuring the whole spectrum of absorbance including a few thousand wavelengths, this approach uses only two wavelengths.

The OEC is an important diagnostic tool that could be deployed in drug research and development associated with treatment of diseases (e.g., sickle cell anemia), athletic performance, physiological status assessment, as well as exploitation in various fields involving hypoxic environments (e.g., high altitude acclimatization).

A blood-derivative or non-blood sample capable of transporting (e.g., binding and releasing) oxygen can be used for generating the OEC in the aforementioned research and development areas and in various aforementioned fields. For example, such a sample can include a liquid that includes a suspension of purified bovine or other red blood cells capable of transporting oxygen, as well as artificial blood that includes a special liquid, a suspension of vesicles, or other carriers capable of transporting oxygen.

It is therefore desirable to provide a robust system, apparatus, and method, which not only overcome the limitations of current systems and methodologies, and thus provide a paradigm shift in OEC measurement, requiring a significantly smaller sample (e.g., blood sample), providing responsive thermo-control of the sample, and allowing measurement of the absorbance spectrum and phosphorescence of the sample, but which also significantly accelerates and simplifies application in drug development, athletic performance, physiological status assessment, and exploitation in a host of other uses.

### SUMMARY

In accordance with an embodiment, there is disclosed a system to test a sample capable of transporting oxygen and including at least a phosphorescence dye. In accordance with another embodiment, there is also disclosed an apparatus to test a sample capable of transporting oxygen and including at least a phosphorescence dye. The apparatus includes an illumination device, a first light collector, a second light collector, and a microcontroller, while the system includes the apparatus and a computing device.

The illumination device is configured to generate a first light pulse for a first time period and a plurality of second light pulses each for a second time period, the first light pulse and the second light pulses capable of illuminating the sample.

The first light collector is configured to collect a plurality of absorbance spectrum measurements from the sample illuminated by the first light pulse.

The second light collector is configured to collect a plurality of phosphorescence measurements emitted by the phosphorous dye of the sample illuminated by the plurality of second light pulses, each phosphorescence measurement being collected during a third time period after a corresponding one of the second light pulses.

The microcontroller is configured to generate an absorbance spectrum average based on the plurality of absorbance spectrum measurements, and further configured to generate a phosphorescence average based on the plurality of phosphorescence measurements.

The computing device is configured to calculate a partial pressure of oxygen (PO2) based on the phosphorescence average and calculate an oxygen saturation of hemoglobin (SatHb) based on the absorbance spectrum average, and the computing device is further configured to plot a point represented by SatHb, PO2 on an oxygen equilibrium curve.

In some cases, the illumination device can include a first light source and a second light source. The first light source can be configured to generate the first light pulse, and the second light source can be configured to generate the plurality of second light pulses.

In some cases, the illumination device can include a light combiner configured to spatially combine the first light pulse of the first light source and the second pulses of the second light source along an optical axis.

In some cases, the light combiner can be a beam splitter configured to allow the first light pulse to pass along the optical axis and further configured to reflect the second light pulse along the optical axis.

In some cases, the first light collector can be positioned along the optical axis in order to collect the plurality of absorbance spectrum measurements, and the second light collector can be positioned at an angle with respect to the optical axis in order to collect the plurality of phosphorescence measurements, wherein the angle can be in a range of about 20° to about 120°. In particular, the angle can be an acute angle of about 60°.

In some cases, the first light pulse can be in a range of 450 nm to 650 nm, and the second light pulse can be in a range of 620 nm to 635 nm.

In some cases, the first light collector can include an optical aperture configured to receive a light transmitted through the sample illuminated by the first light pulse, and a spectral detector can detect the plurality of absorbance spectrum measurements from the sample. The optical aperture can be an optical fiber, while the spectral detector can be a spectrometer.

In some cases, the second light collector can include a phosphorescence detector configured to collect the plurality of phosphorescence measurements emitted by the phosphorescence dye. The phosphorescence dye can have an absorbance in a range 637 nm±10 nm, and an emission in a range 813±20 nm.

In some cases, the apparatus and/or system can further include a heating sample stage that is configured to maintain the sample at a predetermined temperature.

In some cases, the apparatus and/or system can include a position sample stage configured to translate a capillary with the sample along a capillary axis and/or an optical axis.

In some cases, the capillary with the sample is configured to be translated along the capillary axis to an initial position at a predetermined distance from the meniscus of the sample with respect to the optical axis for a first cycle of measurements, and the capillary with the sample is further configured to be translated along the capillary axis by a predetermined distance from the initial position for a subsequent cycle of measurements. In some alternative cases, the capillary with the sample is configured to be translated along the capillary axis to an initial position of the meniscus of the sample with respect to the optical axis for measurements of a plurality of cycles, and a gas flow is configured to be directed toward the meniscus of the sample for the measurements of the plurality of cycles.

In some cases, the sample is a blood sample or a non-blood sample. The non-blood sample can be a liquid that includes a suspension of purified bovine or other red blood cells capable of transporting oxygen, or artificial blood that includes a special liquid, a suspension of vesicles, or other carriers capable of transporting oxygen.

In accordance with a further embodiment, there is provided a method of testing a sample capable of transporting oxygen and including at least a phosphorescence dye, wherein the method includes: generating via an illumination device a first light pulse for a first time period and a plurality of second light pulses each for a second time period, the first light pulse and the second light pulses capable of illuminating the sample; collecting via a first light collector a plurality of absorbance spectrum measurements from the sample illuminated by the first light pulse; collecting via a second light collector a plurality of phosphorescence measurements emitted by the phosphorous dye of the sample illuminated by the plurality of second light pulses, each phosphorescence measurement being collected during a third time period after a corresponding one of the second light pulses; generating via a controller an absorbance spectrum average based on the plurality of absorbance spectrum measurements, and further generating a phosphorescence average based on the plurality of phosphorescence measurements; calculating via a computing device a partial pressure of oxygen (PO2) based on the phosphorescence average and calculating an oxygen saturation of hemoglobin (SatHb) based on the absorbance spectrum average, and further plotting via the computing device a point represented by SatHb, PO2 on an oxygen equilibrium curve.

In some cases, the method can further include generating the first light pulse via a first light source of the illumination device, and generating the plurality of second light pulses via a second light source of the illumination device.

In some cases, the method can further include spatially combining the first light pulse of the first light source and the second light pulses of the second light source along an optical axis.

In some cases, the method can further include passing the first light pulse along the optical axis and reflecting the second light pulse along the optical axis via a beam splitter.

In some cases, the method can further include positioning the first light collector along the optical axis in order to collect the plurality of absorbance spectrum measurements, and positioning the second light collector at an angle with respect to the optical axis in order to collect the plurality of phosphorescence measurements, wherein the angle is in a range of about 20° to about 120°. In particular, the angle can be an acute angle of about 60°.

In some cases, the first light pulse can be in a range of 450 nm to 650 nm, while the second light pulse can be in a range of 620 nm to 635 nm.

In some cases, the method can further include: receiving a light transmitted through the sample illuminated by the first light pulse via an optical aperture of the first light collector; and detecting the plurality of absorbance spectrum measurements from the sample via a spectral detector of the first light collector. The optical aperture can be an optical fiber, and the spectral detector is a spectrometer.

In some cases, the method further includes collecting the plurality of phosphorescence measurements emitted by the phosphorous dye via a phosphorescence detector of the second light collector.

In some cases, the phosphorescence dye can have an absorbance in a range 637 nm±10 nm, and an emission in a range 813±20 nm.

In some cases, the method can further include maintaining the blood at a predetermined temperature via a heating sample stage.

In some cases, the method can further include translating a capillary with the sample along a capillary axis and/or an optical axis via a position sample stage.

In some cases, the method can include translating via the position sample stage the capillary with the sample along the capillary axis to an initial position at a predetermined distance from the meniscus of the sample with respect to the optical axis for a first cycle of measurements, and further translating via the position sample stage the capillary with the sample along the capillary axis by a predetermined distance from the initial position for a subsequent cycle of measurements. In some alternative cases, the method can include translating via the position sample stage the capillary with the sample along the capillary axis to an initial position of the meniscus of the sample with respect to the optical axis for measurements of a plurality of cycles, and flowing a gas via a tube directed toward the meniscus of the sample for the measurements of the plurality of cycles.

In some cases, the sample is a blood sample or a non-blood sample. The non-blood sample can be a liquid that includes a suspension of purified bovine or other red blood cells capable of transporting oxygen, or artificial blood that includes a special liquid, a suspension of vesicles, or other carriers capable of transporting oxygen.

These and other purposes, goals, and advantages of the present application will become apparent from the following detailed description of example embodiments read in connection with the accompanying drawings.

### BRIEF DESCRIPTION OF THE DRAWINGS

Some embodiments are illustrated by way of example and not limitation in the figures of the accompanying drawings in which:

FIG. 1 illustrates a block diagram of an example system configured to illuminate a capillary containing a blood sample, periodically measure and average an optical absorbance spectrum (also referenced herein as “absorbance spectrum”) as well as measure and average kinetics of phosphorescence decay (also referenced herein as “phosphorescence”) of the blood sample as the blood sample is deoxygenated, continually calculate a partial pressure of oxygen (PO2) and an oxygen saturation of hemoglobin (SatHb) based on the average measurements, and finally generate the Oxygen Equilibrium Curve (OEC) 900 based on the calculations;

FIG. 2 illustrates a block diagram of an apparatus (instrument) configured to optically measure and average the absorbance spectrum and the average phosphorescence of the blood sample as input to the generation of the OEC in accordance with the example system of FIG. 1;

FIG. 3 illustrates a block diagram of a microcontroller configured to control the apparatus (instrument) in measuring the absorbance spectrum and phosphorescence of the blood sample, and further configured to average the absorbance spectrum and phosphorescence as input to the computing device for the generation of the OEC, in accordance with the example system of FIG. 1;

FIG. 4 illustrates a block diagram of an example apparatus in accordance with FIGS. 1-3;

FIG. 5 illustrates a flowchart of an example method of charging a capillary with a preparation of a blood sample for measurement of the absorbance spectrum and phosphorescence via one of several deoxygenation methods in accordance with the example system of FIGS. 1-4;

FIG. 6 illustrates a flowchart of an example method executed by the microcontroller in measuring and averaging the absorbance spectrum and the phosphorescence of the blood sample in accordance with FIGS. 1-5;

FIG. 7 illustrates a flowchart of an example method executed by a computing device in calculating PO2 and SatHb and associated plotting on the OEC based respectively on the average absorbance spectrum and the average phosphorescence, collected via one of several deoxygenation methods in accordance with FIGS. 1-6;

FIG. 8 illustrates an example timing diagram of the apparatus (instrument) operation in accordance with FIGS. 1-7;

FIG. 9 illustrates an example user interface (UI) generated by the computing device in connection with measuring and computing the average absorbance spectrum and the average phosphorescence, as well as generating an OEC using respectively calculated PO2 and SatHb, via one of several deoxygenation methods in accordance with FIGS. 1-7; and

FIG. 10 illustrates a block diagram of an example general computer system capable of performing any methods or computer-based functions in accordance with FIGS. 1-9.

### DETAILED DESCRIPTION

A system, apparatus, and method of testing a sample (e.g., blood sample) to generate an oxygen equilibrium curve are described herein. While the following description addresses examples of testing a blood sample, it should be understood that the system, apparatus, and method are equally applicable to testing a non-blood sample capable of transporting (e.g., binding and releasing) oxygen, such as a liquid that includes a suspension of purified bovine or other red blood cells capable of transporting oxygen, as well as artificial blood that includes a special liquid, a suspension of vesicles, or other carriers capable of transporting oxygen. In the following description, for the purposes of explanation, numerous specific details are set forth in order to provide a thorough understanding of example embodiments or aspects. It will be evident, however, to one skilled in the art, that an example embodiment may be practiced without all of the disclosed specific details.

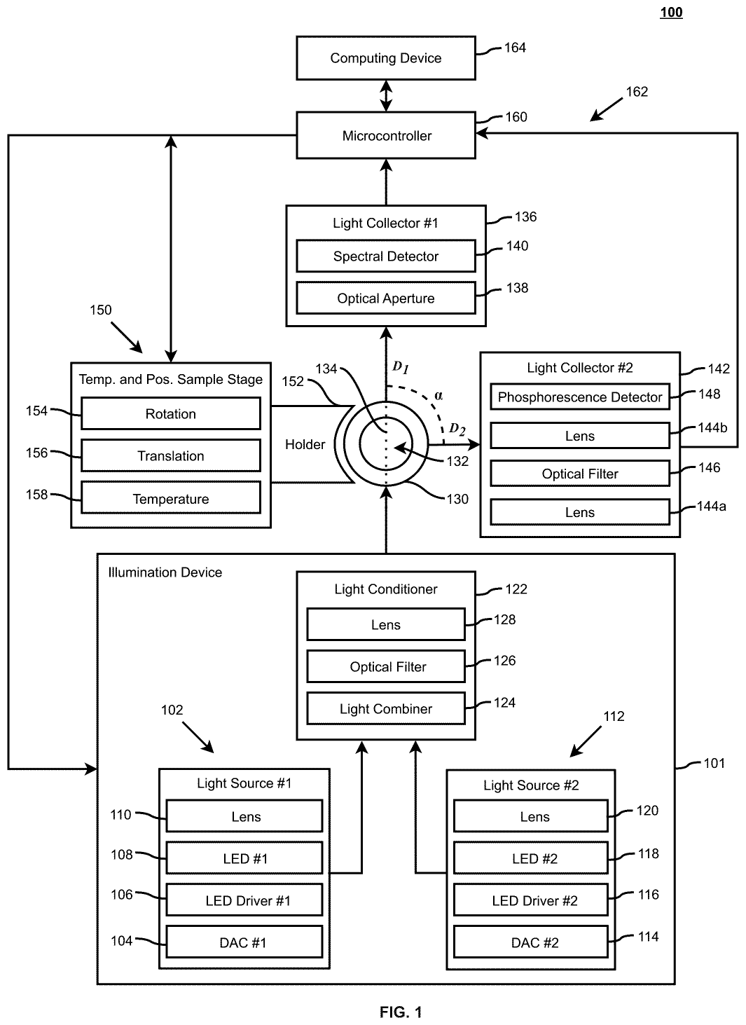

FIG. 1 illustrates a block diagram of an example system 100 configured to illuminate a capillary 130 containing a blood sample 132, periodically measure and average an optical absorbance spectrum (also referenced herein as “absorbance spectrum”) as well as measure and average kinetics of phosphorescence decay (also referenced herein as “phosphorescence”) of the blood sample 132 as the blood sample is deoxygenated, continually calculate a partial pressure of oxygen (PO2) and an oxygen saturation of hemoglobin (SatHb) based on the average measurements, and generate the Oxygen Equilibrium Curve (OEC) 900 based on the calculations.

The example system 100 includes an apparatus (instrument) 162 and a computing device 164. The computing device 164 can be connected to the instrument 162 in a wireless configuration (e.g., via one or more of WiFi, WAN, LAN, etc.) or a wired configuration (e.g., via universal serial bus (USB), serial peripheral interface (SPI) bus, recommended standard (RS) including RS 232, 422, 485, controller area network (CAN) bus, etc.), and/or via one or more communication devices, busses, or interfaces (not shown) using conventional or yet to be developed communication standards.

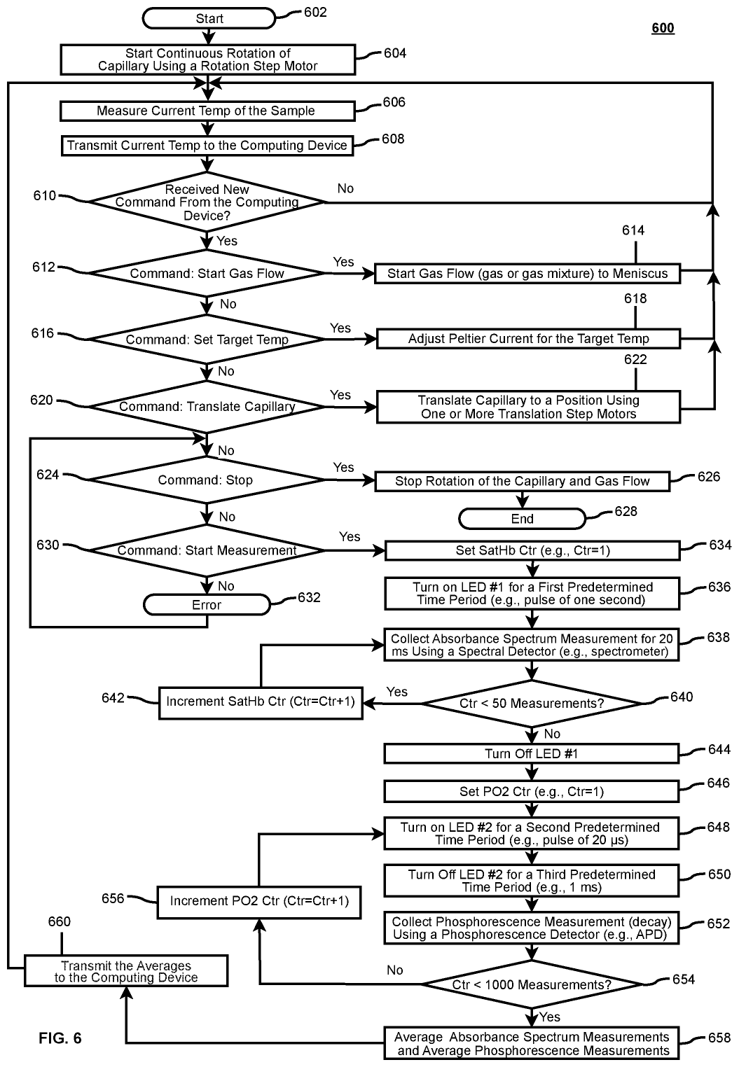

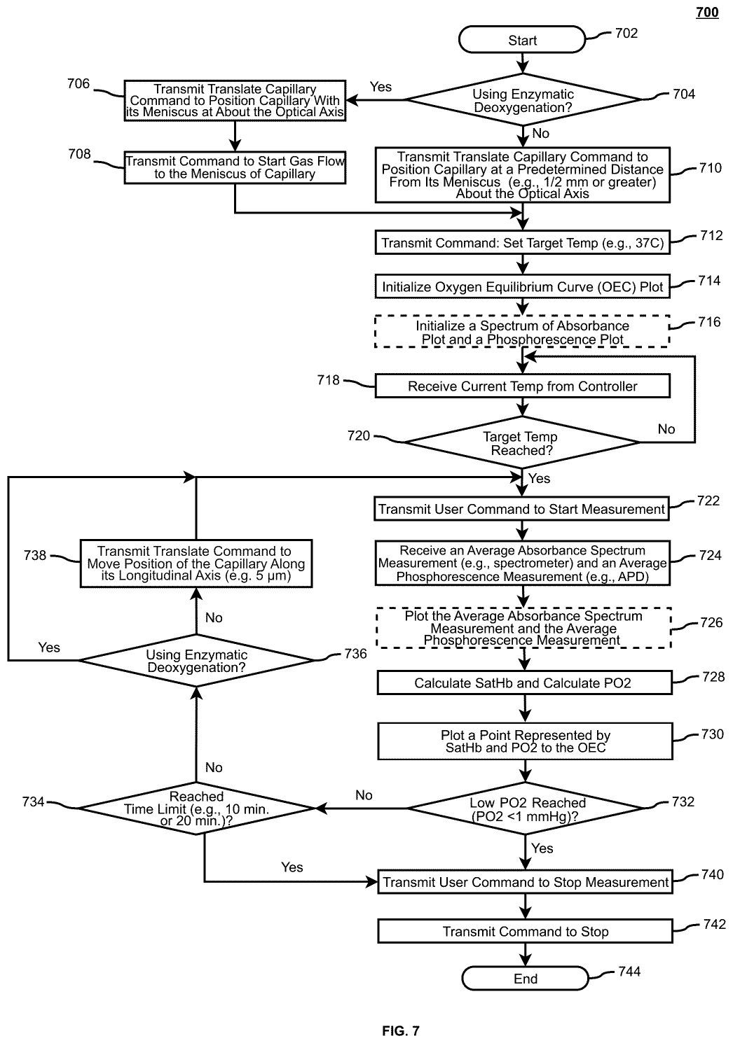

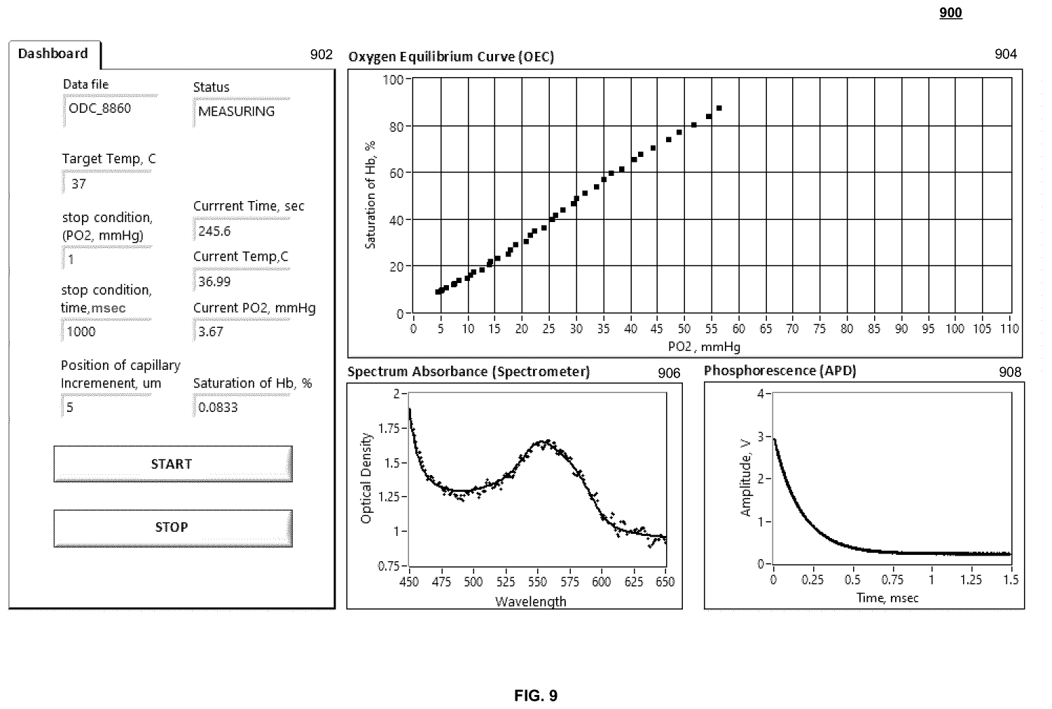

Generally in operation which will be described with much greater detail in reference to the methods illustrated in FIGS. 6 and 7, the computing device 164 instructs or commands the instrument 162 to periodically measure an absorbance spectrum and a phosphorescence of the blood sample 132 in the capillary 130 as the blood sample 132 is deoxygenated, and further compute respective measurement averages of the absorbance spectrum and an phosphorescence. During each such cycle in which the instrument 162 returns the average measurements, the computing device 164 continually calculates PO2 based on the average absorbance spectrum and SatHb based on the average phosphorescence, and plots a point on the OEC that is represented by a coordinate value (SatHb, PO2), as particularly shown in the user interface (UI) in FIG. 9. The measurements, calculations, and associated plotting can continue until one or more conditions are satisfied, such as a predefined threshold of PO2 is reached (e.g., PO2=1 mm Hg) and/or a predefined time limit is reached (e.g., 10 minutes or 20 minutes) from the start. It should be noted that the foregoing measurements can be collected and/or displayed in real-time using commercially available software that can be executed by the computing device 164 (e.g., LabVIEW® available from National Instruments Corporation of Austin, Texas).

The apparatus (instrument) 162 is configured to periodically measure the absorbance spectrum and the phosphorescence of the blood sample 132 in the capillary 130 as the blood sample 132 is deoxygenated, compute respective average measurements of the absorbance spectrum and the phosphorescence, and return the average measurements to the computing device 164. In particular, the instrument 162 includes an illumination device 101, a temperature and position sample stage 150, a first light collector 136, a second light collector 142, and a controller 160. While not shown in FIG. 1, the instrument 162 can have a power source sufficient to power its functionality and allow it to communicate with the computing device 164.

The capillary 130 can be cylindrical and should have a sufficiently small inner diameter that can induce capillary or wicking flow of a blood sample 132 into the capillary 130. For example, the capillary 130 can have an inner diameter of 100 μm or smaller (e.g., 10 μl microcap manufactured by Drummond Scientific Company). The capillary 130 can be fabricated from one or more materials, including borosilicate, clear fused quartz, synthetic fused silica, etc. The foregoing types of capillaries are commercially available from VitroCom of Mountain Lake, New Jersey, as well as Drummond Scientific Company of Broomall, Pennsylvania. As an example, VitroCom offers cylindrical capillaries that have the following inner diameters of 0.05 mm, 0.10 mm, 0.15 mm, 0.20 mm, 0.30 mm, 0.40 mm, 0.50 mm, 0.60 mm, 0.70 mm, 0.80 mm, 0.90 mm, 1.00 mm, 1.50 mm, 2.00 mm, and the like, as well as the following lengths of 100 mm, 300 mm, 600 mm, and the like.

Generally, the illumination device 101 is configured to combine light from at least two sources and project the combined light illuminating the blood sample 132 in the capillary 130 such that the absorbance spectrum and the phosphorescence of the blood sample can be measured as a result of the light being incident on the blood sample 132 of the capillary 130. In particular, the illumination device 101 includes a first light source 102, a second light source 112, and a light conditioner 122.

The first light source 102 is configured to generate a continuous spectrum of light in a range of wavelengths that are capable of being absorbed by hemoglobin in the blood sample 132 (e.g., a range of 450 nm to 650 nm). The first light source 102 includes a digital-to-analog converter (DAC) 104, a light emitting diode (LED) driver 106, an LED 108, and a lens 110.

The DAC 104 is configured to set a power level of the LED 108 for a plurality of pulses of light to be generated by the LED 108, as will be described in greater detail hereinbelow with reference to FIG. 6. For example, the power level that is used could be set to 1 watt-5 watts, and in some preferred cases the DAC 104 sets the power level to 3 watts. For example, the microcontroller 160 can set a code (e.g., power level) of the LED driver 106 such as via a voltage setting module 340, wherein the code corresponds to a voltage associated with powering the LED 108. The voltage thus governs the power level of the LED 108 in generating the associated plurality of light pulses.

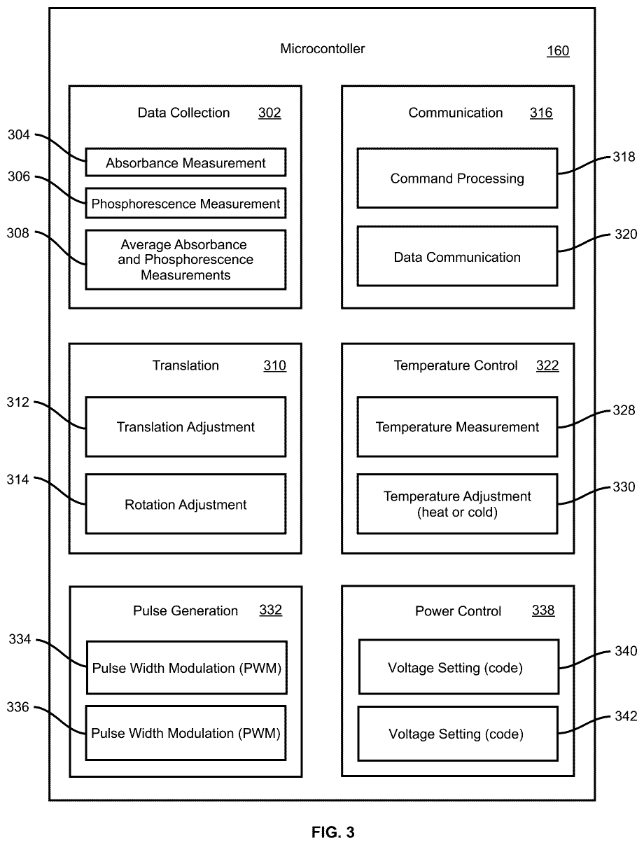

The LED driver (e.g., MOSFET-based electronics) 106 is configured to drive the LED 108 in generating the plurality pulses (e.g., pulses of approximately 20 microseconds (μs)) when operated by a respective plurality of pulses generated by the microcontroller 160, such as via a pulse width modulation (PWM) module 330 of the microcontroller 160 illustrated in FIG. 3.

Accordingly, the LED 108 generates the aforementioned continuous spectrum of light in pulses at the power level set by the DAC 104 and based on the pulses as generated by the LED driver 108. The lens 110 is configured to project the light generated by the LED 108 to the light conditioner 122, in particular to the combiner 124.

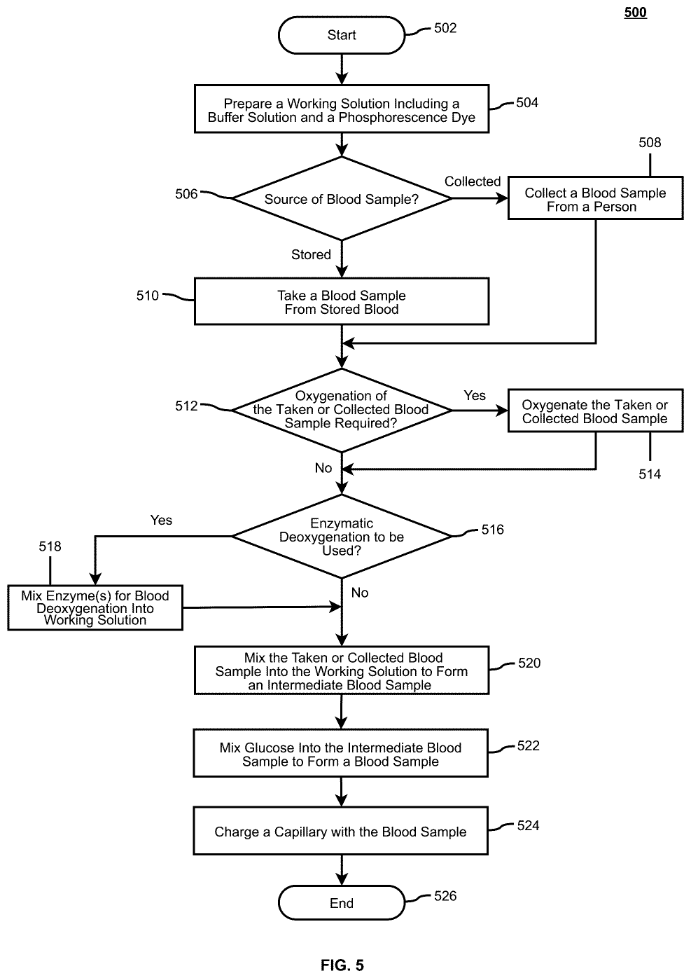

The second light source 112 is configured to generate a relatively monochromatic light (e.g., a bandwidth of 10 nm, 20 nm, or 100 nm) in a range suitable to excite phosphorescence of a phosphorescence dye mixed into the blood sample 132, as will be described hereinbelow in detail with reference to FIG. 5. For example, in a case of PdG4 phosphorescence dye produced by Oxygen Enterprises Ltd., in which the absorption is 637 nm±10 nm and the emission is 813 nm±20 nm, the monochromatic light generated by the first light source 102 would be in a range of 620 nm-635 nm, thus approximating the absorption of this phosphorescence dye. Another phosphorescence dye can be used and the monochromatic light generated by the first light source 102 can thus be selected to approximate that dye's absorbance. The second light source 112 includes a digital-to-analog converter (DAC) 114, a light emitting diode (LED) driver 116, an LED 118, and a lens 120.

The DAC 114 is configured to set a power level of the LED 118 for a plurality of pulses of light to be generated by the LED 118, as will be described in greater detail hereinbelow with reference to FIG. 6. For example, DAC 114 can set the power level in a similar fashion as described hereinabove with reference to DAC 104, wherein the DAC 114 sets the power level to 1 watt-5 watts, and in some preferred cases the power level is set to 3 watts. In a similar fashion to DAC 104, the microcontroller 160 can set a code (e.g., power level) of the LED driver 116 such as via a voltage setting module 342, wherein the code corresponds to a voltage associated with powering the LED 118. The voltage thus governs the power level of the LED 118 in generating the associated plurality of light pulses.

The LED driver (e.g., MOSFET-based electronics) 116 is configured to drive the LED 118 in generating the plurality pulses (e.g., pulses of approximately one second) when operated by a respective plurality of pulses generated by the microcontroller 160, such as via a pulse width modulation (PWM) module 332 of the microcontroller 160 illustrated in FIG. 3.

Accordingly, the LED 118 generates the aforementioned monochromatic light in pulses at the power level set by the DAC 114 and based on the pulses as generated by the LED driver 118. The lens 120 is configured to project the light generated by the LED 118 to the light conditioner 122, in particular light to the combiner 124.

The light conditioner 122 is configured to combine the first light generated by the first light source 102 and the second light source 112, filter the light, and project the light to the blood sample 132 in the capillary 130. The light conditioner 122 includes a light combiner 124, an optical filter 126, and a lens 128.

The light combiner 124 is configured to combine the first light emitted in first pulses by the first light source 102 and the second light emitted in second pulses by the second light source 112 such that the lights are effectively combined in space into a third light with respect to the optical axis 134 of the instrument 162. For clarity purposes, the third light emitted by the light combiner 124 would be either the first light as emitted by the first light source 102 or the second light as emitted by the second light source 112, both being aligned with respect to the optical axis 134 of the instrument 162. In particular, the light conditioner, including the light combiner 124, optical filter 126, and lens 128 are aligned to the optical axis 134 of the instrument 162.

The optical filter 126 is configured to filter the third light emitted by the light combiner 124. For example, a high-pass filter (HPF) can be applied to filter or clean the third light of frequencies that contaminate the third light. For example, as the light combiner 124 would nominally emit a third light in a range of 450 nm-650 nm (e.g., first light emitted by the first LED 108) and would nominally emit a third light in a range of 620 nm-635 nm (e.g., second light emitted by the second LED 118), the optical filter 126 can be selected to clean the emitted third light of frequencies that might contaminate the third light, e.g., filtering certain wavelengths or ranges of wavelengths with respect to one or more of the nominally emitted ranges (e.g., filtering wavelength longer than 670 nm).

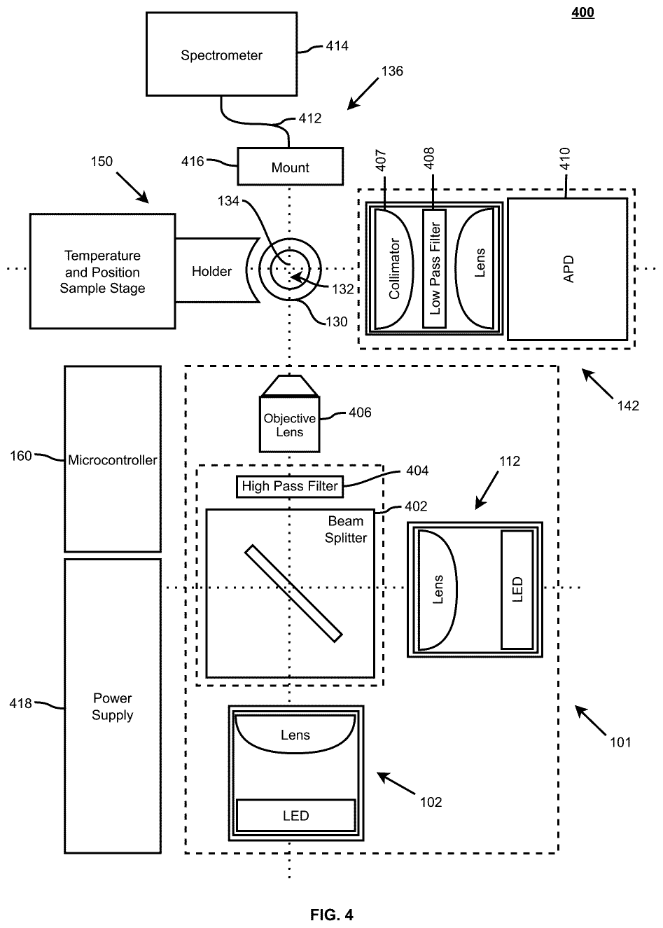

The lens 128 is configured to project and focus the combined third light such that it is imaged along the optical axis 134 on the blood sample 132 at approximately the capillary axis 231 of the capillary 130. In some cases, the lens 128 can be an objective lens that can be selected to have a certain magnification (e.g., 2×, 4×, 5×, or 10× magnification, etc.), as will be described in greater detail with reference to FIG. 4.

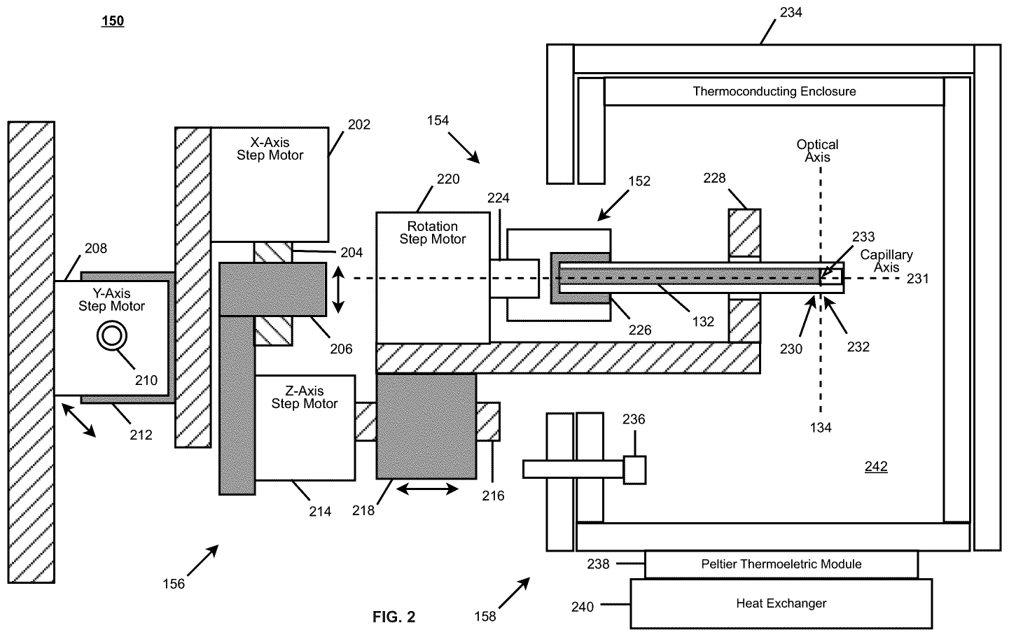

Generally, the temperature and position sample stage 150 of the instrument 162 is configured to rotate the capillary 130 about the capillary axis 231, translate the capillary 130 in relation to the optical axis 134, and further to maintain a temperature of the blood sample 132 in the capillary 130. More specifically, the temperature and position sample stage 150 is configured to initially align the capillary 130 along the capillary axis 231 of the instrument 162, and also initially align the capillary 130 to one of the initial optical axes 203, 232, which is based on a type of method used for deoxygenation (i.e., enzymatic or non-enzymatic deoxygenation), as particularly described in greater detail with reference to FIG. 2. The temperature and position sample stage 150 includes a holder 152, a rotation component 154, a translation component 156, and a temperature measurement/adjustment component 158.

The holder 152 is configured to receive, secure, and align the capillary 130 along the capillary axis 231 of the instrument 162. The holder 152 is further configured to be rotated about the capillary axis 231 and translated along capillary axis 231 to align the capillary 130 with an optical axis 134, e.g., initially aligned with one of the optical axes 203, 232.

The rotation component 154 is configured to rotate the capillary 130 (i.e., holder 152 with the capillary 130) about the capillary axis 231 at a certain speed (e.g., one turn per minute) in order to promote mixing of the blood sample 132. Other speeds of rotation are of course possible. What is important to note is that the speed of rotation should be sufficient to promote mixing of erythrocyte suspension (i.e., red blood cells) in the blood sample 132.

The translation component 156 is configured to translate the capillary 130 along x-y-z directions such that it can be aligned to the capillary axis 231 as well as to the optical axis 134, so that the blood sample 132 can be probed at the optical axis 134 along potentially different distances of the capillary axis 231, as will be described in greater detail hereinbelow with reference to FIGS. 6 and 7.

The temperature measurement/adjustment component 158 is configured to measure the temperature of the blood sample 132 (e.g., measuring the temperature of the blood sample 132 itself, the temperature of the capillary 130, or the ambient temperature about the capillary 130 charged with blood sample 132) and adjust the temperature to a given temperature (e.g., 37° C.), as will be described in greater detail hereinbelow with reference to FIGS. 6 and 7.

Generally, the first light collector 136 of the instrument 162 is positioned at a distance D1 (e.g., 0.1 mm to 1 mm) to the capillary 130 along the optical axis 134 and is configured to detect an absorbance spectrum of the blood sample 132 in the capillary 130. As already described hereinabove, the first light from the first light source 102 has a spectrum in the range of 450 nm-650 nm, and thus the first light (i.e., also referenced as a third light emitted from the light conditioner 122) can be efficiently absorbed by hemoglobin. However, it should be noted that the first light is only partially absorbed by hemoglobin in the optical capillary 130, and remaining light (non-absorbed first light) passes through the blood sample 132 and the capillary 130. The first light collector 136 includes an optical aperture 138 and a spectral detector 140.

The optical aperture 138 is configured to receive a portion of the remaining light that passes through the blood sample 132 and the capillary 130. The portion of the remaining light passed through the capillary 130 carries information about the absorbance spectrum of Hemoglobin. The optical aperture is small, such as for example, being a size of an optical fiber (e.g., having a diameter less than 100 micrometers (μm)). For example, the diameter of the optical aperture 138 can be 10 μm, 20 μm, or 50 μm, and in some preferred cases the diameter can be 62.5 μm. The optical aperture 138 can be an optical fiber or can be formed in a surface (e.g., a surface of the spectral detector 140) so that the portion of the remaining light passed through the blood sample 132 and the capillary 130 can be detected by the spectral detector 140. In particular, the optical aperture 138 is positioned and configured to enable the portion of the remaining light passed through the blood sample 132 and the capillary 130 to be received close to a center of the capillary 130, e.g., taking about 10% of an inner diameter of the capillary 130. In this case, the cylindrical shape of the capillary 130 can thus be approximated as a parallelepiped and Lambert-Beer Law can be applied in order to estimate a concentration of oxygenated and deoxygenated hemoglobin.

The spectral detector 140 is configured to detect an absorbance spectrum of the portion of the remaining light that passes through the optical aperture 138. A plurality of absorbance spectrum measurements is collected and transmitted to the controller 160, wherein the measurements are averaged for ultimate transmission to the computing device 162, as described in greater detail hereinbelow with reference to FIGS. 6 and 7.

Generally, the second light collector 142 of the instrument 162 is disposed at an angle (a) to the optical axis 134, and is configured to detect phosphorescence emitted from the blood sample 132 of the capillary 130. The angle α of the second light collector to the optical axis 134 can be any angle from about 20° to about 120°, and in some preferred cases can be about 60°. The angle α is chosen to enable a close or a closest positioning of the light collector 142, such as at a distance D2, because such positioning improves signal strength. It should be noted that other particular angles are of course possible based on testing requirements. As already described hereinabove, the example PdG4 phosphorescence dye absorbs the second light of 620 nm-635 nm and produces a phosphorescence emission of about 813 nm±20 nm. Other dyes that might be used can have different absorption and emission profiles. The second light collector 142 includes lenses 144a, 144b, optical filter 146, and phosphorescence detector 148.

The lens 144a is configured to collect a phosphorescence emission that is emitted by the phosphorescence dye of the blood sample 132. In particular, the lens 144a has a short focal length (e.g., corresponding to distance D2) so that a sufficient amount of the phosphorescence emission can be collected. The focal length (D2) can be in a range 5 mm-25 mm and a diameter of the lens 144a can thus be in a range 5 mm-25 mm. The ratio of the diameter to the focal length (D/F) should be close to one (1) in order to permit collection of light as much as possible. It is possible to use an aspheric lens for this purpose. A phosphorescence emission of at least 9-10 watts (e.g., at a peak of the phosphorescence emission) can be expected to be collected by the lens 144a. The phosphorescence detector 148 (e.g., APD 410) should be sufficiently sensitive to detect such a phosphorescence.

The optical filter 146 is configured to filter the phosphorescence emission collected by the lens 144a. For example, a low-pass filter (LPF) can be applied to filter or clean the collected phosphorescence emission. In the illustrated example of the PdG4 phosphorescence dye, the emitted phosphorescence is in a range of 813±20 (i.e., 793 nm-833 nm), and thus the optical filter 146 can be selected to filter wavelengths shorter than 750 nm, or in an alternative wavelengths shorter than 715 nm. For other phosphorescence dyes, the optical filter 146 can be appropriately selected to filter out frequencies that contaminate the phosphorescence emission. It should be noted that the methodology involved in phosphorescence detection is based on excitation of a phosphorescence emission using a relatively strong light (excitation) that has a first range of wavelengths, and further based on detection of a relatively very weak light (emission) that has a different second range of wavelengths. In this regard, a ratio of intensities between excitation and emission can be as large as a million times. Accordingly, it is very important to filter out light that is related to excitation from light that is related to emission. In this regard, it is preferred to use a combination of a low-pass optical filter (LPF) after pulse generation and before illumination of the sample 132 in the capillary 130 (e.g., optical filter 126) coupled with a long-pass filter (LPF) after phosphorescence emission and before detection of the phosphorescence emission (e.g., filter 146).

The lens 144b is configured to focus the phosphorescence emission collected by the lens 144a on a phosphorescence detector 148, as described in greater detail hereinbelow. In particular, a combination of the lenses 144a and 144b forms a so-called optical condenser. The optical condenser (e.g., formed by lenses 144a and 144b) makes it possible to efficiently collect the phosphorescence emission and to focus the collected phosphorescence emission on the phosphorescence detector 148 (e.g., APD 410). In this regard, the benefit of the optical condenser is that phosphorescence emission is a parallel beam between the lenses 144a and 144b, making it possible to position the optical filter 146 therebetween.

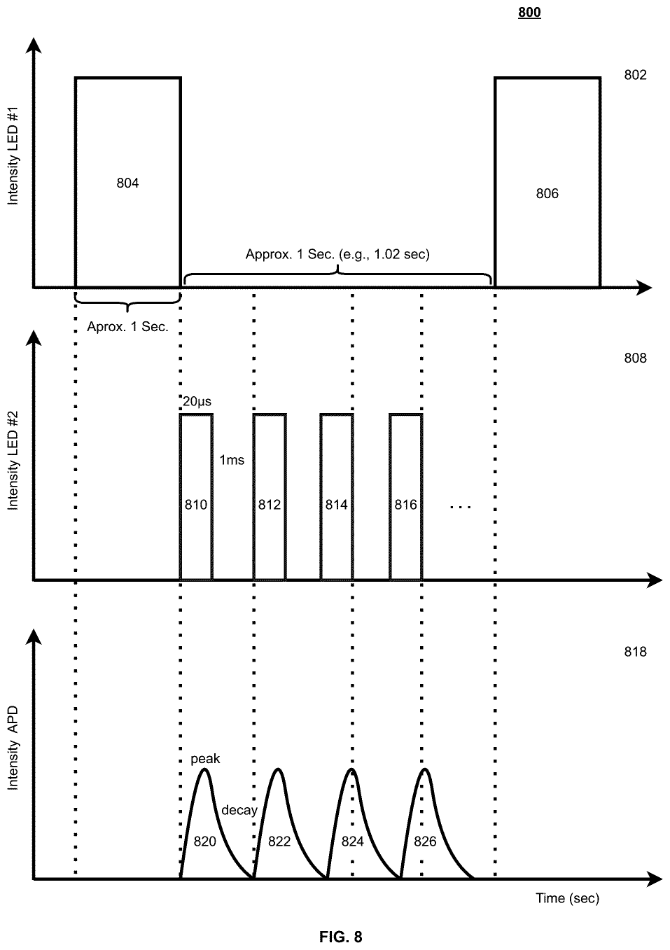

The phosphorescence detector 148 is configured to detect the phosphorescence emitted from the blood sample 132, as collected and focused by lenses 144a, 144b and filtered by the optical filter 146 positioned therebetween. A plurality of phosphorescence measurements are collected, wherein each phosphorescence measurement represents the kinetics of phosphorescence decay (e.g., a plurality of decay measurements after a peak of the phosphorescence emission, as shown in panel 818 of FIG. 8), and the collected phosphorescence measurements are then transmitted to the controller 160, wherein the phosphorescence measurements, each representing a plurality of phosphorescence decay measurements, are averaged for ultimate transmission to the computing device 162, as described in greater detail hereinbelow with reference to FIGS. 6 and 7.

In view of the foregoing, the microcontroller 162 receives the plurality of absorbance spectrum measurements as detected by the first light collector 136 resulting from the first light pulses generated by the first light source 102, and then averages these measurements to generate an average absorbance spectrum measurement. The microcontroller 162 further receives the plurality of phosphorescence measurements detected by the second light collector 142 as a result of the second light pulses generated by the second light source 112, and then averages these measurements to generate an average phosphorescence measurement (e.g., average kinetics of phosphorescence decay). In those cases where the phosphorescence detector 148 (e.g., APD 408) generates analog phosphorescence measurements (e.g., each phosphorescence measurement representing a plurality of phosphorescence decay measurements), the decay measurements of each of these phosphorescence measurements can be digitized by the microcontroller 162 before averaging all of the digitized phosphorescence measurements (e.g., averaging the decay measurement at each position of the kinetics decays among the plurality of phosphorescence measurements) for eventual transmission of the average phosphorescence measurement to the computing device 164. Thereafter, the microcontroller 162 transmits the average absorbance spectrum measurement and the average phosphorescence measurement to the computing device 164 for generation and presentation of the OEC.

FIG. 2 illustrates a block diagram of a temperature and position sample stage 150 of the apparatus (instrument) 162 configured to measure and average the absorbance spectrum and measure and average the phosphorescence of the blood sample as input to the generation of the OEC in accordance with the example system of FIG. 1.

As described hereinabove, the temperature and position sample stage 150 is configured to initially align the capillary 130 along the capillary axis 231 of the instrument 162, and also initially align the capillary 130 to the optical axis 134 at an initial position 230, 232, which is based on a type of method used for deoxygenation (i.e., enzymatic deoxygenation or non-enzymatic deoxygenation), as particularly described in greater detail with reference to FIG. 2. The temperature and position sample stage 150 includes a holder 152, a rotation component 154, a translation component 156, and a temperature measurement/adjustment component 158.

The holder 152 is configured to receive, secure, and align the capillary 130 along the capillary axis 231 of the instrument 162. In particular, the holder 152 includes a soft collar 226 and a support 228. In some cases, a user can insert the capillary 130 into the holder 152, while in other cases the capillary 130 can be inserted using an automatic capillary changer (not shown), which can be beneficial for mass screenings (e.g., drug discovery research). The soft collar 226 is disposed at approximately a first end of the capillary 130 and is configured to securely yet safely support the capillary 130 in its rotation and translation as described herein. In particular, the holder 152 is configured to be rotated about the capillary axis 231 and translated along capillary axis 231 to align the capillary 130 with an optical axis 134 as shown in FIG. 1, e.g., at one of the two positions 230, 232 as shown in FIG. 2, depending on the method used for deoxygenation. The support 228 is positioned at approximately a second end of the capillary 130 and at a distance from the collar 226, and configured to support capillary 130 such that the collar 226 in combination with the support 228 can precisely maintain the capillary 130 along the capillary axis 231 during its rotation and translation as described herein.

The rotation component 154 is configured to rotate the capillary 130 about the capillary axis 231 at a certain speed (e.g., one turn per minute) in order to promote mixing of the blood sample 132. It is noted that other speeds of rotation are possible. What is important is that the rotation be sufficient to promote mixing of the erythrocyte suspension (i.e., red blood cells) in the blood sample 132. The rotation component 154 includes a rotation step motor 220 and a rotating shaft 224 to which the holder 152 is secured. Upon start of the instrument 162, the microcontroller 160 instructs the rotation step motor 220 to continuously rotate the shaft 224 at a certain speed (e.g., one turn per minute). The speed depends on the viscosity of the sample, and can be predetermined (or preset) in the microcontroller 160 or can be provided to the microcontroller 160 by the computing device 164.

The translation component 156 is configured to translate the capillary 130 along x-y-z directions such that it can be aligned to the capillary axis 231 as well as to the optical axis 134 shown in FIG. 1 at one of two positions (e.g., positions 230, 232), so that the blood sample 132 can be probed at the optical axis 134 potentially at different distances along the capillary axis 231, as will be described in greater detail hereinbelow with reference to FIGS. 6 and 7.

In particular, the translation component 156 includes constituent x-axis, y-axis, and z-axis translation components configured to translate the capillary 130 respectively along x, y, and z axes. The x-axis translation component includes an x-axis step motor 202, a screw 204, and slider 206. In operation, upon an issued command by the controller 160, the step motor 202 turns the screw 204 and allows the slider 206 to translate along the x-axis (e.g., translate to a certain position or a given distance from a current position).

The y-axis translation component includes a y-axis step motor 208, a screw 210, and slider 212. In operation, upon an issued command by the controller 160, the step motor 208 turns the screw 210 and allows the slider 210 to translate along the y-axis (e.g., translate to a certain position or a given distance from a current position).

The z-axis translation component includes a z-axis step motor 214, a screw 216, and slider 218. In operation, upon an issued command by the controller 160, the step motor 214 turns the screw 216 and allows the slider 218 to translate along the z-axis (e.g., translate to a certain position or a given distance from a current position).

Accordingly, the translation component 156 can translate the capillary 130 along x-y-z axes such that the capillary 130 can be aligned to the capillary axis 231 as well as to the optical axis 134 shown in FIG. 1 at one of two positions (e.g., positions 230, 232), such that the blood sample 132 can be probed at the optical axis 134 potentially at different distances along the capillary axis 231.

The temperature measurement/adjustment component 158 is configured to measure the temperature of the blood sample 132 (e.g., measuring the temperature of the blood sample 132 itself, the temperature of the capillary 130, or the ambient temperature about the capillary 130 charged with the blood sample 132) and adjust the temperature to a given temperature (e.g., 37° C.), as will be described in greater detail hereinbelow with reference to FIGS. 6 and 7. The temperature measurement/adjustment component 158 includes a thermo-conducting enclosure 234, a temperature sensor 236, a Peltier thermoelectric module 238, and a heat exchanger 240.

The thermo-conducting enclosure 234 is configured to enclose the capillary 130 and further configured to conduct heat or cold from the thermoelectric module 238, so as to maintain a certain constant temperature of the ambient air 242 in the enclosure and about the capillary 130, and thus maintain the temperature of the blood sample 132 in the capillary 130.

The temperature sensor 236 is configured to measure the current temperature of the ambient air 242 and to transmit the measured temperature to the microcontroller 160. In addition to or instead of the temperature sensor 236, a temperature senor can be provided that is configured to measure a temperature of the capillary 130 and/or a temperature of the blood sample 132 in the capillary 130. For example, a thermocouple (not shown) can be positioned on or inside the capillary 130.

The Peltier thermoelectric module 238 is connected to the thermo-conducting enclosure 234 and is configured to heat or cool the enclosure such that ambient air 242 in the enclosure 234 can be respectively heated or cooled in order to maintain a certain temperature (e.g., 37° C.). In particular, a first surface of Peltier module 238 is connected to the enclosure 234, and a second surface is connected to the heat exchanger 240. In operation, upon issuance of a temperature command from the computing device 164 and an associated issuance of an instruction from the microcontroller 160 to adjust the temperature, the Peltier module 238 is configured to heat or cool the first surface so as to heat or cool the ambient air 242 in the enclosure 234 to a target temperature, and reciprocally thereto further configured to conduct cold or heat to the heat exchanger 240 via the second surface. The heat exchanger 240 is configured to dissipate the cold or heat conducted by the Peltier thermoelectric module 238.

FIG. 3 illustrates a block diagram of a microcontroller 160 configured to control the apparatus (instrument) 162 in measuring the absorbance spectrum and phosphorescence of the blood sample 132, and further configured to average the absorbance spectrum and phosphorescence as input to the computing device 164 for the generation of the OEC 904 in accordance with the example system of FIG. 1.

The microcontroller 160 can be an application specific integrated circuit (ASIC), micro-electromechanical system (MEMS), or a system on chip (SOC) capable of performing the functionality and/or functions of data collection 302, translation 310, communication 316, temperature control 322, pulse generation 328, and power control 338, as described herein and with reference to FIGS. 6 and 7. The microcontroller 160 can further include a processing unit (microprocessor) and a memory that stores instructions executable by the processing unit to perform operations associated with data collection 302, translation 310, communication 316, temperature control 322, pulse generation 328, and power control 338, as described herein and with reference to FIGS. 6 and 7.

Data collection 302 includes functionality (e.g., functions) performed or executed by the microcontroller 160 associated with collecting a plurality of absorbance spectrum measurements 304 via the spectrum detector 140, collecting phosphorescence measurements 304 via the phosphorescence detector 148, and averaging the absorbance spectrum measurements and the phosphorescence measurements 308, as described herein and with reference to FIGS. 6 and 7.

Translation 310 includes functionality (e.g., functions) performed or executed by the microcontroller 160 associated with x-y-z translation adjustment via x-axis, y-axis, and z-axis translation components 156 configured to translate the capillary 130 respectively along x, y, and z axes, as well as rotation adjustment 314 of the capillary 130 about the capillary axis 231 via the rotation component 154, as described hereinabove with reference to FIGS. 2, 6, and 7.

Communication 316 includes functionality (e.g., functions) performed or executed by the microcontroller 160 associated with command processing 318 as issued by the computing device 162 in relation to pulse generation, power control, gas provision, data collection, translation, and temperature control, as well as data communication 320 of computed averages of the absorbance spectrum measurements and the phosphorescence measurements for OEC generation by the computing device 164, as described herein and with reference to FIGS. 6 and 7.

Temperature control 322 includes functionality (e.g., functions) performed or executed by the microcontroller 160 associated with temperature measurement 324 via temperature sensor 236 and temperature adjustment (heat or cold) 326 of the thermo-conducting enclosure 234 via the Peltier thermoelectric module 238 and the respective dissipation of cold or heat via the heat exchanger 240, as described herein with reference to FIGS. 2, 6, and 7.

Pulse generation 328 includes functionality (e.g., functions) performed or executed by the microcontroller 160 associated with pulse width modulation (PWM) 330 for generating first pulses of first light emitted by the first light source 102, and PWM 332 for generating second pulses of light emitted by the second light source 112, as described herein and with reference to FIGS. 1, 6, and 7.

Power control 334 includes functionality (e.g., functions) performed or executed by the microcontroller 160 associated with voltage setting (mode) 336 for generating the first pulses of first light emitted by the first light source 102 at a certain power level, and voltage setting (mode) 338 for generating the second pulses of light emitted by the second light source 112 at the certain power level, as described herein and with reference to FIGS. 1, 6, and 7.

The microcontroller 160 can further include functionality (e.g., functions) that can be performed or executed to start and stop (e.g., via a valve) flow of gas toward the meniscus 233 of the blood sample 132 along the capillary axis 231 (e.g., via a tube) in order to deoxygenate the blood sample, according to one of the deoxygenation methods described herein.

FIG. 4 illustrates a block diagram of an example apparatus (instrument) 400 in accordance with FIGS. 1-3. Instrument 400 can be similar to or different than the instrument 162, and can includes the following particular components.

In the illumination device 101, the light combiner 122 can be a beam splitter 402, which emits a third light by passing the first light from the first light source 102, and reflecting the second light from the second light source 112, such that the lights are effectively combined in space (as opposed to time) with respect to the optical axis 134 of the instrument 162. As described hereinabove, the third light emitted by the beam splitter 402 would be either the first light as emitted by the first light source 102 or the second light as emitted by the second light source 112, both being aligned with respect to the optical axis 134 of the instrument 162 by the beam splitter 402.

As already described hereinabove, the optical filter 126 can be a high-pass filter (HPF) 404, which is configured to filter the third light emitted by the beam splitter 402. The HPF 404 can be applied to filter the third light, e.g., clean the third light of frequencies that might contaminate the third light. For example, as the beam splitter 402 would nominally emit a third light in a range of 450 nm-650 nm (e.g., first light emitted by the first LED 108) and would nominally emit a third light in a range of 620 nm-635 nm (e.g., second light emitted by the second LED 118), the HPF 404 can be selected to clean the emitted third light of frequencies that might contaminate the third light, e.g., filtering certain wavelengths or ranges of wavelengths with respect to one or more of the nominally emitted ranges (e.g., filtering wavelengths longer than 670 nm).

As described hereinabove, the lens 128 can be an objective lens 406 selected to have a certain magnification, such as for example a 2×, 4×, 5×, or 10× magnification, etcetera, in order to magnify the combined third light projected and focused along the optical axis 134 on the blood sample 132 at approximately the capillary axis 231 of the capillary 130.

In the first light collector 136, the optical aperture 138 could be an optical fiber 412 (e.g., having a diameter less than 100 μm) held by a mount 416. In some cases, the optical fiber 412 can be a bare optical fiber and the mount 410 can be a standard or custom mechanical holder configured to hold the bare optical fiber. In other cases, the optical aperture 138 can be a flexible fiber connector (e.g., SubMiniature-A connector (SMA), Subminiature Push-on (SMP) connector, etc.) that houses the optical fiber and enables its connection to the spectral detector 140 (e.g., a spectrometer 414 as described hereinbelow). In the case of terminated fibers or terminated flexible fiber connectors, the mechanical holder should keep a first end of the bare fiber or the fiber of the connector aligned with the optical axis 134 and open to the capillary 130 (e.g., at a distance D1 of 0.1 mm-1 mm), whereas the second end of bare fiber or the fiber of the connector is connected to spectral detector 140. The optical fiber 412 is configured to receive and communicate a portion of the remaining light that passes through the blood sample 132 and the capillary 130 to the spectral detector 140, and is a perfect optical aperture (diaphragm) which can minimize the amount of scattered light that can potentially interfere with the phosphorescence measurements of the second light collector 142 (e.g., APD 410).

In alternative fiber-free implementations (not shown), the capillary 130 and spectral detector 140 (e.g., spectrometer 414 as described hereinbelow) can be optically coupled (e.g., using optical element(s) such as one or more lenses). In particular, instead of the optical fiber 412, the optical aperture 138 can be an entrance slit or aperture (e.g., having a similar or different dimension or diameter as described herein with reference to fiber implementations) directly in the spectral detector 140, wherein the spectral detector is positioned right after the capillary 130 (e.g., mounted by an appropriate holder). In such cases, the spectral detector 140 can include or support the optical element(s) such as one or more lenses in order to communicate the remaining light that passes through the blood sample 132 and the capillary 130 through the entrance slit or aperture for detection by the spectral detector 140 (e.g., spectrometer 414).

Moreover, the spectral detector 140 of the first light collector 136 can be a spectrometer 414, which is configured to detect an absorbance spectrum of light that passes through the optical aperture 138 (e.g., via optical fiber 412 or aperture in fiber-free embodiments). Suitable spectrometers can include Avantes and Ocean Optics that are commercially available spectrometers, or one or more other spectrometers yet to be developed.

In the second light collector 142, the optical filter 146 can be a low-pass filter (LPF) 408 applied to filter or clean the collected phosphorescence emission from the blood sample 132. As described hereinabove, if the example PdG4 phosphorescence dye is used, the emitted phosphorescence is in a range of 813±20 (i.e., 793 nm-833 nm), and thus the LPF 408 can be selected to filter wavelengths shorter than 750 nm, or in an alternative wavelengths shorter than 715 nm. For other phosphorescence dyes, the LPF 408 can be appropriately selected to filter out frequencies that might contaminate the phosphorescence light.

Moreover, the phosphorescence detector 148 of the second light collector 142 can be an avalanche photodiode sensor (APD) 410 and the lens 144a can be a collimator 407. As a result of illuminating the blood sample 132 with the second light source 112, the blood sample 132 emits a phosphorescence (a weak near-infrared light) which can be efficiently collected and collimated by the collimator 407, filtered by the LFP 408, focused by the lens 144b (shown in FIG. 1), and detected with the APD 410. Other suitable electro-optical detectors can be used, such as a photodiode, a photomultiplier, a complementary metal-oxide semiconductor (CMOS), a charge-coupled device (CCD) camera, or a similar detector.

The instrument 400 includes a power supply to supply 418 so that the instrument can perform the functionality and/or functions described herein. The power supply 418 can be a battery power supply (e.g., for a portable instrument 400), a USB or other conventional power supply plugged into a standard wall outlet.

FIG. 5 illustrates a flowchart of an example method 500 of charging a capillary with a preparation of a blood sample for measurement of the absorbance spectrum and phosphorescence via one of several methods in accordance with the example system of FIGS. 1-4.

The method 500 starts at operation 502, where the components and elements that are necessary to charge the capillary 130 are obtained or available. At operation 504, there is prepared a working solution that includes a buffer solution and a phosphorescence dye. The buffer solution can be PBS×1, HEMOX, or any other suitable buffer solution. In some preferred cases, a HEMOX buffer solution with pH=7.4 at 37° C. can be used. Whereas the buffer solution is used to keep the blood of the eventual blood sample 132 stable, the profile of the phosphorescence dye is used to select parameters associated with light generation of the light source 112 as well as phosphorescence detection of the light collector 142. Moreover, the phosphorescence dye is selected so that absorbance of hemoglobin in blood does not interfere with the absorbance of the phosphorescence dye.

At operation 506, a determination is made as to the source of the blood for the blood sample. If it is determined that the blood will be collected, then at operation 508 a blood sample is collected from a person (e.g., finger prick). However, if it is determined that blood will be taken from storage, then at operation 510, a blood sample is taken from pre-collected stored blood. The method 500 of charging the capillary and subsequent testing of the blood sample using the system 100 can use as little as 1 microliter (μl), or 2 μl of blood. The sample of blood (e.g., drop of blood) can be transferred from a stored standard tube or collected from the finger prick.

At operation 512, a further determination is made as to whether oxygenation of the blood sample will be required. The blood sample should be oxygenated to a level of saturation of hemoglobin with oxygen that is at least 80%. If it is determined that oxygenation will be required, then at operation 514 the blood sample is oxygenated to a certain level of saturation of hemoglobin and the method 500 continues at operation 516. A standard oxygenation machine (e.g., a machine using oxygen permeable membranes or bubbling action) can be used to oxygenate the blood sample. Generally, oxygenation is not a problem when the blood sample is collected from a finger prick and used promptly, as the blood is highly oxygenated. However, stored blood or an erythrocyte concentrate is significantly deoxygenated and thus should be oxygenated before its use.

If it is determined that oxygenation is not required, then at operation 516 a determination is made as to whether enzymatic deoxygenation will be used or not used. It should be noted that this selection represents the two (2) deoxygenation methods for the measurements as described herein.

If it is determined that enzymatic deoxygenation is not to be used, the method continues at operation 520. However, if it is determined that enzymatic deoxygenation is to be used, then at operation 518 one or more enzymes (e.g., one or more enzymatic liquids or powders) are mixed into the working solution. The one or more enzymes can be glucose oxidase and can optionally include catalase. Thereafter, the method continues at operation 520.

At operation 520, the taken or collected blood sample is mixed into the working solution, forming an intermediate blood sample. At operation 522, in order to activate the enzymes associated with the deoxygenation, glucose is mixed into the intermediate blood sample thus forming the blood sample 132. It should be noted that the concentration of the enzymes and the glucose in the blood sample 132 can be tuned to a time of complete deoxygenation being achieved in approximately 10 minutes. The foregoing concentration can thus allow testing (e.g., measurements and generation of OEC) to be completed in approximately 10 minutes. One or more other concentrations of the enzymes and the glucose can be selected in order to achieve different deoxygenation times and thus different testing times.

Lastly, at operation 524 the capillary 130 is charged with the blood sample 132 (e.g., using capillary action). Thereafter, the method 500 ends at operation 526, where the capillary 130 with the blood sample 132 is ready for use in the instrument 162.

It should be noted that all or some of the foregoing components (e.g., blood sample, buffer solution, enzymes, and/or glucose, etc.) can be charged into the capillary 130, frozen or freeze-dried in the capillary 130, and then thawed or diluted when necessary in future use. Similarly, the foregoing components can instead be charged into an auxiliary vial (e.g., a PCR tube of about 200 μl), frozen or freeze-dried in the vial, thawed or diluted and then charged into the capillary 130 when necessary in future use. It should further be noted that adopting pre-filled capillaries can be extremely beneficial for applications in the field using portable variants of the system 100, particularly portable apparatus (instrument) 160.

FIG. 6 illustrates a flowchart of an example method 600 executed by the microcontroller 160 in measuring and averaging the absorbance spectrum and the phosphorescence of the blood sample 132 via several methods in accordance with FIGS. 1-5.

The method 600 starts at operation 602, where a user secures capillary 130 charged with the blood sample 132, as prepared in accordance with the method of FIG. 5, into the holder 152 of the instrument 162, and further turns on the apparatus (instrument) 162.

At operation 604, continuous rotation of capillary 130 is started using a rotation step motor 220, as particularly illustrated in FIG. 2. As described hereinabove, the rotation should be sufficient to promote mixing of erythrocyte suspension (i.e., red blood cells) in the blood sample 132 (e.g., one revolution per minute).

At operation 606, a current temperature of the blood sample 132 is measured via temperature sensor 236. It should be noted that the temperature measurement of the blood sample 132 is carried out by measuring the temperature of the ambient air 242 in the thermo-conducting enclosure 234, as particularly illustrated in FIG. 2, or directly measuring the temperature of the thermo-conducting enclosure 234. At operation 608 the current temperature is transmitted to the computing device 164, where it could be displayed on the graphical user interface (UI) 900 to the user.

At operation 610, the microcontroller awaits receipt of a new command from the computing device 164, and processes the received command to perform specific functionality associated with that command, as described in greater detail hereinbelow.

If it is determined that the received command is “start gas flow” as illustrated at 612, then at operation 614 gas flow (e.g., a pure gas such as nitrogen, helium, argon, or oxygen, air or another gas, or a mixture of one or more of the foregoing gasses) is started toward the meniscus 233 in order to deoxygenate the blood sample, according to one of the deoxygenation methods described herein. The method 600 then continues to operation 610 to await another command.

If it is determined that the received command is “set target temp” as illustrated at operation 616, then at operation 618 the Peltier current of the Peltier thermoelectric module 238 is adjusted to the provided target temperature. As illustrated on the UI 900 in FIG. 9, the user can adjust the target temperature to 37° C. causing the issuance of the command to set the target temperature. The method 600 then continues to operation 610 to await another command.

If it is determined that the received command is “translate capillary” as illustrated at operation 620, then at operation 622 the capillary is translated to a provided position or by a provided offset distance using one or more steps motors 202, 208, 214, as illustrated in FIG. 2. The provided position can indicate an initial position 230, 232 of the capillary 132 with respect to the optical axis 134, i.e., whether it the position 232 of the meniscus 233 or a position 230 at a distance from the meniscus 233. Moreover, the offset distance (e.g., 5 μm) can be provided from a current position (e.g., from the initial position 230 or a subsequent position) and can indicate a position at which a further measurement is to be taken.

If it is determined that the received command is “stop” as illustrated at operation 624, then at operation 626 the continuous rotation of the capillary 130 is stopped, and the flow of gas is also stopped. Thereafter, the method ends at operation 628.

If it is determined that the received command is “start measurement” as illustrated at operation 630, then the microcontroller 160 performs the operations 634-660 as described below. Multiple “start measurement” commands can be received from the computing device 164 until certain condition(s) is/are met, as described in greater detail with reference to FIG. 7. However, when an unrecognized or a corrupted command is received and/or determined (e.g., corruption that might result from or during transmission), the microcontroller generates an error 632, which might be transmitted to the computing device 164 for display on the UI 900, and in response to the error 632 issues a command “stop” that executes the operations 626, 628 in connection with the stop command 624.

The following operations 634-660 are performed in connection with the command “start measurement” determined at operation 630. In particular, operations 634-644 are performed for the collection of a plurality of absorbance spectrum measurements during one cycle, or iteration through the method 600 based on one of a plurality of “start measurement” commands received from the computing device 164, as will be described in greater detail with reference to FIG. 7. Accordingly, at operation 634 a hemoglobin saturation counter SatHb Ctr is set (e.g., Ctr=1). At operation 636, the first light source 102 or more particularly the first LED 108 is turned on for a first a predetermined time period (e.g., a pulse of one second). At operation 638, an absorbance spectrum measurement is collected for 20 ms using a spectral detector 140 (e.g., spectrometer 408). At operation 640, a determination is made as to whether the counter indicates that fewer than 50 measurements were collected (e.g., Ctr<50). If it is determined that fewer measurements were collected, the saturation counter is incremented (Ctr=Ctr+1) at operation 642, and the method 600 continues at operation 638 with the collection of the next absorbance spectrum measurement for 20 ms. The method 600 iterates operations 638-642 until it is determined that 50 absorbance spectrum measurements are collected at operation 640. It should be noted that 50 measurements at 20 ms for each measurement equals the duration of approximately one second (i.e., 50*20 ms=1 sec.), the length of the light pulse. Thereafter, the first light source 102 (i.e., first LED 108) is turned off at operation 644.

Moreover, operations 646-656 are performed for the collection of a plurality of phosphorescence measurements during one cycle, or iteration through the method 600 based on one of a plurality of “start measurement” commands received from the computing device 164, as will be described in greater detail with reference to FIG. 7. Accordingly, at operation 646 an oxygen pressure counter PO2 Ctr is set (e.g., Ctr=1). At operation 648, the second light source 112 or more particularly the second LED 118 is turned on for a second predetermined time period (e.g., a pulse of 20 microseconds (p)). At operation 650, the second light source 112 (i.e., second LED 118) is turned off for a third predetermined time period (e.g., 1 millisecond (ms)) to allow for the collection of a phosphorescence measurement. At operation 652, the phosphorescence measurement is thus collected using a phosphorescence detector 148 (e.g., APD 416). It should be noted that each phosphorescence measurement represents the kinetics of phosphorescence decay (e.g., a plurality of decay measurements taken after a peak of the phosphorescence emission, as shown in panel 818 of FIG. 8). In some preferred cases, 1000 decay measurements can be taken for each phosphorescence measurement, whereas a different number of decay measurements is also possible based on various testing requirements. At operation 654, a determination is made as to whether the pressure counter indicates that fewer than 1000 phosphorescence measurements were collected (e.g., Ctr<1000). If it is determined that fewer phosphorescence measurements were collected, the pressure counter is incremented (Ctr=Ctr+1) at operation 656, and the method 600 continues at operation 648 with the collection of the next phosphorescence measurement.

The method 600 iterates operations 648-656 for collection of the phosphorescence measurements until it is determined that 1000 phosphorescence measurements (each phosphorescence measurement obtaining 1000 decay measurements) are collected at operation 652. It should be noted that the duration of 1000 phosphorescence measurements equals approximately one second (i.e., [1000*20 μs=0.02 sec.]+[1000*1 ms=1 sec.]=1.02 sec.), a similar duration to the cycle of absorbance measurements. One million decay measurements are taken in approximately one second (e.g., 1 MHz). Accordingly, the total measurement time of absorbance spectrum and the phosphorescence measurements can be approximately two seconds (e.g., 2.02 sec.). It should be noted that the cycles associated with the absorbance spectrum and phosphorescence measurements do not have to be approximately the same in duration. For example, the third time period during the collection of each phosphorescence measurement can be 1 ms, 2 ms, 3 ms, or another duration, wherein in the case of 3 ms the duration of 1000 measurements equals approximately three seconds (i.e., [1000*20 μs=0.02 sec.]+[1000*3 ms=3 sec.]=3.02 sec.). Accordingly, the total measurement time of absorbance spectrum and the phosphorescence measurements can be approximately four seconds (e.g., 4.02 sec.).

At the conclusion of the absorbance spectrum and phosphorescence measurements during one cycle associated with the command “start measurement” from the computing device 164, the absorbance spectrum measurements and the phosphorescence measurements are respectively averaged at operation 658. At operation 660, the absorbance spectrum average and the phosphorescence average are transmitted to the computing device 164 for computation of SatHb and PO2 in connection with the generation of the OEC.

In view of the foregoing, the measurement time for the absorbance spectrum is chosen to be just enough to saturate the spectral detector 140 with light. This saturation also depends on the intensity of the first light from the first light source 102. The choice as to number of averages for the absorbance spectrum is based on a trade-off between precision and a number of points generated on OEC. The bigger the number of computed averages the greater the precision in determining the saturation, but the fewer points that would be potentially generated on the OEC. Exactly the same can be said about choice of the measurement time, number of measurements, and averages of the phosphorescence measurements, i.e., each of the measurements representing kinetics of phosphorescence decay.

The method 600 continues at operation 606 to measure the current temperature of the blood sample 132, and at operation 608 transmits the temperature to the computing device 164 for display on the UI 900, as particularly illustrated in FIG. 9.

FIG. 7 illustrates a flowchart of an example method 700 executed by a computing device 164 in calculating PO2 and SatHb and associated plotting on the OEC based respectively on the average absorbance spectrum and the average phosphorescence, collected via one of several the deoxygenation methods in accordance with FIGS. 1-6.

The method 700 starts at operation 702, where the user has secured capillary 130 charged with the blood sample 132, as prepared in accordance with the method of FIG. 5, into the holder 152 of the instrument 162, and further turned on the apparatus (instrument) 162.

At operation 704, a determination is made as to whether enzymatic deoxygenation is being used. Such a determination can be made based on whether a user has charged the capillary with blood sample 130 containing an enzymatic solution or not as described hereinabove in reference to FIG. 5, and particularly based on whether the user entered a position of the capillary increment (e.g., 5 μm or another increment) in the UI 900 of the computing device 164.

If it is determined that enzymatic deoxygenation is to be used, at operation 706 a translate capillary command is transmitted to the microcontroller 160 to translate the capillary 130 along the capillary axis 231 to an initial position 232 of the meniscus 233 (e.g., blood-air interface) that is to be positioned at about the optical axis 134. At operation 708, a start gas command is transmitted to the microcontroller 160 to start gas flow to the meniscus 233 of capillary 130. However, if it is determined that enzymatic deoxygenation is not to be used, at operation 710 a command is transmitted to the microcontroller 160 to translate the capillary 130 along the capillary axis 231 to a position 230 a predetermined distance from the meniscus 233 (e.g., ½ mm or greater distance from the meniscus 233), that is to be positioned at about the optical axis 134. For example, in some embodiments the predetermined distance is 2 mm or greater.

At operation 712, a set target temp command is transmitted to the microcontroller 160 in order to set the target temperature (e.g., 37° C.) of the blood sample 132, as particularly described in reference to FIG. 6. It should be noted that the target temperature for the various measurements described herein can be set to a different temperature (e.g., 36.6° C.).

At operation 714, an oxygen equilibrium curve (OEC) plot is initialized, wherein a plurality of points each being represented by SatHb, PO2 (e.g., relation SatHb(PO2)) will be plotted thereon for each cycle of measurement to define an OEC over time as the blood sample 132 is deoxygenated.

At operation 716, a spectrum of absorbance plot as well as phosphorescence plot (kinetics of phosphorescence day) can be optionally initialized, wherein the average spectrum of absorbance measurements as well as the average phosphorescence measurements (each representing kinetics of phosphorescence decay) can be plotted for each cycle of measurements to define the spectrum of absorbance and phosphorescence over time as the blood sample 132 is deoxygenated.

At operation 718, the computing device 164 receives a current temperature from microcontroller 160, such as for example shown in the UI 900 illustrated in FIG. 9.

At operation 720, a determination is made as to whether the target temperature (e.g., 37° C.) has been reached within a predetermined precision (e.g., 0.01° C., 0.015° C., 0.02° C., or another precision value). In some instances, the target temperature can be reached in several minutes (e.g., 5 min.-10 min.) depending on starting temperature (e.g., room temperature) and a heating rate via the Peltier thermos-electric module 238, as particularly illustrated in FIG. 2. In some cases, the target temperature can be reached in a shorter or a longer time period. If it is determined that the target temperature has not been reached, the method 700 continues at operation 716. In one example, the start button on the UI 900 in FIG. 9 can be disabled until the target temperature is reached. Alternatively, start button may be enabled and it can be up to user as to whether to press the start button before the target temperature is reached.

If it is determined that the target temperature has been reached at operation 720, the method 700 iterates operations 722-738 until one or more termination conditions are satisfied. At operation 722, the user transmits a start measurement command to the microcontroller 160 in order to start measurements, as described in greater detail with reference to FIG. 6. At operation 724, the computing device 164 receives from the microcontroller 160 an average absorbance spectrum measurement (e.g., collected via spectrometer) and an average phosphorescence measurement (e.g., collected via APD). At operation 726, the average absorbance spectrum measurement and the average phosphorescence measurement (kinetics of phosphorescence decay) can be optionally plotted, for example as shown at respective plots 906, 908 in the UI 900 illustrated in FIG. 9.

At operation 728, SatHb and PO2 are calculated based on the received absorbance spectrum and phosphorescence measurement averages. As to the calculation of the oxygen saturation of hemoglobin (SatHb), the absorbance spectrum is fit with known components including spectra of hemoglobin (Hb), oxygenated hemoglobin (OxyHb), and Methemoglobin (MetHb), and possibly other components using method of singular value decomposition (SVD), or any other suitable method. Generally, SVD is a widely used method that plays a role in data preprocessing for machine learning. It is mostly used to filter out noise from the data, reduce dimensionality of the data, and fit the data. In particular, SVD is configured to find a best combination of components (e.g., Hb, MetHb and OxyHb) that fit an experimental spectrum. SVD routine is the standard part of many mathematical libraries of languages such as C, C++, Java, MATLAB, LabVIEW, Python, as well as other programing languages. In this way, a composition of the blood sample 132, or a concentration of the foregoing components (e.g., Hb, MetHb and OxyHb) is known for every cycle of the measurements. Accordingly, oxygen saturation of hemoglobin (SatHb) can be calculated with the following equation: SatHb=OxyHb/(Hb+OxyHb).

As to the calculation of the oxygen pressure (PO2), the average phosphorescence measurement (e.g., averaging all kinetics of phosphorescence decay measured in one measurement cycle) is fit with an exponential decay. In particular, a well know Levenberg-Marquardt algorithm can be implemented for the fitting. An example of the kinetics of phosphorescence decay is shown in plot 908 of the UI 900 illustrated in FIG. 9. The vertical axis is amplitude of the phosphorescence (e.g., signal) in volts, while the horizontal axis is in milliseconds, so that the kinetics of the phosphorescence decay for a phosphorescence measurement of one cycle are illustrated over one second. Plot 908 can be generated for each phosphorescence measurement. The rate of the decay is directly connected to pressure according to a calibration curve. The calibration curve is individualized for the phosphorescence dye used and is available from the manufacturer of the phosphorescence dye. The calibration is generally accomplished via a lookup in a computed table or an equation that computes PO2 for every rate of decay.

At operation 730, the computing device 164 plots a point represented by the calculated SatHb and PO2 to the OEC, as particularly illustrated in OEC plot 904 of UI 900 illustrated in FIG. 9.

At operation 732, a determination is made as to whether a first stop condition is satisfied, e.g., has PO2 reached a predetermined low PO2 value (e.g., PO2<1 mm Hg). While the low PO2 value used is set to 1 mm Hg, it should be noted that another PO2 value can be used as the first stop condition. If the first condition is satisfied at operation 732, then the method 700 continues at operation 740 and the computing device awaits a user's input to stop. Such input can be received from the user with a stop button as illustrated on the dashboard 902 of the UI 900 illustrated in FIG. 9. Upon user's selection of the stop button, at operation 742 the computing device 164 transmits a stop command to the microcontroller 160 in order stop rotation of the capillary 130 and stop gas flow, if any, to the meniscus 233.

If the first stop condition is not satisfied at operation 732, then at operation 734 a determination is made as to whether a second stop condition is satisfied, e.g., has the elapsed time since the start of the measurements reached a certain time limit (e.g., 10 min. or 20 min.). Elapsed time is shown on the dashboard 902 of the UI 900 illustrated in FIG. 9. For example, if the blood sample 132 in the thermo-conducting enclosure 234 is at temperature (e.g., 37° C.) and the averages of the spectrum and phosphorescence are generated approximately in two seconds, then processing the measurements in order to complete the generation of the OEC can take approximately 10 min. However, if the blood sample 132 in the thermo-conducting enclosure 234 is not temperature (e.g., 37° C.) and the averages are generated in approximately four seconds, then processing the measurements in order to complete the generation of the OEC can take approximately 20 min. Other limitations or constraints may dictate the expected time limit, such as a time limit between 10 min and 20 min, or higher than 20 mins. What is important to note is that a time limit is selected as to when it is expected that the measurements are completed and/or the OEC generated.

Accordingly, if it is determined that the second stop condition is satisfied at operation 734, then the method 700 continues with performance of operations 740 and 742, as already described hereinabove with reference to the determination of whether the first condition has been satisfied at operation 732. In particular, upon receiving at operation 440 a user's input to stop, at operation 742 the computing device 164 transmits a stop command to the microcontroller 160 in order stop rotation of the capillary 130 and stop gas flow, if any, to the meniscus 233.

However, if it is determined that the second stop condition is not satisfied at operation 734, then the method 700 continues at operation 736 to determine whether enzymatic deoxygenation is being used. This can be determined for example based on whether the user entered a position of the capillary increment (e.g., 5 μm or another increment) in dashboard 902 of the UI 900 illustrated in FIG. 9.

If it is determined that enzymatic deoxygenation is not being used at operation 736, then at operation 738 the computing device 164 transmits a translate capillary command to the microcontroller 160 in order to translate the capillary 130 along the capillary axis 231 to a position defined by the current position minus the capillary increment (e.g., 5 μm or another increment) positioned at about the optical axis 134. The method 700 continues to iterate operations 722-738 until either the first stop condition or the second stop condition has been satisfied.

However, if it is determined that the enzymatic deoxygenation is being used at operation 736, then the method 700 continues to iterate operations 722-736 until either the first stop condition or the second stop condition is satisfied. In other words, the capillary 130 is not translated and remains with its meniscus positioned at about the optical axis 134.

In view of the foregoing description, if one of the first condition and the second condition is satisfied, the operations 740 and 742 are then performed as already described hereinabove. Thereafter, the method 700 ends at operation 744.

FIG. 8 illustrates an example timing diagram 800 of the apparatus (instrument) 612 operation in accordance with FIGS. 1-7.

As depicted in panel 802, a cycle of measurements starts with a pulse 804 of approximately 1 sec. generated by the microcontroller 160 destined to the first light source 102 to turn on the first LED 108 in connection with the measurement of the absorbance spectrum of the blood sample 132. During this one-second pulse, the spectral detector 140 (e.g., spectrometer 414) collects 50 spectrum measurements, and then transmits these measurements to the microcontroller 160 for averaging.

As depicted in panel 808, after the first LED 108 is turned off, the cycle of measurements continues when the microcontroller 160 turns on the second LED 118 for a train of short pulses each of approximately 20 μs 810-816, etc. of monochromatic light, which pulses are separated by a longer period (e.g., approximately 1 ms) when the second LED 118 is turned off. These pulses excite the phosphorescence dye and the emitted phosphorescence is detected by the phosphorescence detector 148 (e.g., APD 410) at rate of approximately 1 MHz, as already described herein (e.g., 1000 phosphorescence measurements with each representing kinetics of phosphorescence decay). Therefore, the kinetics of every phosphorescence measurement includes approximately 1000 decay measurements. Each phosphorescence measurement (kinetics) has a shape of exponential decay: exp(−t/τ), where t is time and tau τ is known as a lifetime of the phosphorescence decay or simply a rate of decay. Tau τ has dimension of time and is equal to a time that it takes for the phosphorescence to drop (decay) approximately 2.7 times.

As depicted in panel 818, the phosphorescence detector 148 detects all kinetics 820-826 of phosphorescence decay (e.g., decay measurements that are lower than the peak measurement of phosphorescence) for each of the pulses 810-816, etc. and transmits the phosphorescence measurements to the microcontroller 160 for averaging. During a cycle of approximately one-second (e.g., approximately 1.02 sec.), the phosphorescence detector 148 (e.g., APD 410) collects 1000 phosphorescence measurements representing 1000 decay measurements (e.g., 1 MHz). It should be noted that the lifetime of the phosphorescence (τ) generally depends on the phosphorescence dye that is used, and the lifetime of the phosphorescence can thus be in a range of 0.1 ms to 2 ms, and sometimes longer up to 3 ms. Therefore, the timing of the measurements should be adjusted for the different phosphorescence dyes.

As further depicted in panel 802, the next cycle of measurements starts with a pulse 806 in connection with further absorbance spectrum measurements, and continues with a plurality of pulses (not shown in panel 808) in connection with further phosphorescence measurements. In all, the absorbance spectrum measurements (e.g., 50) measurements) and the phosphorescence measurements (e.g., 1000 decays for each of 1000 phosphorescence measurements) of this cycle are then transmitted to the microcontroller 160 for averaging.

FIG. 9 illustrates an example user interface (UI) 900 generated by the computing device 164 in connection with measuring and computing the average absorbance spectrum and the average phosphorescence, as well as generating an OEC plot 902 using respectively calculated PO2 and SatHb, via one of several deoxygenation methods as described in accordance with FIGS. 1-7.

The user interface includes a dashboard 902, an oxygen equilibrium curve (OEC) plot 904, and optionally the spectrum of absorbance plot 908 and phosphorescence plot 908.

The dashboard 902 includes a first set of parameters (left column) and a second set of parameters (right column). The first set of parameters can be selected by a user in connection with the measurements performed by the system 100. The data file stores various computed measurements (e.g., absorbance spectrum, phosphorescence (kinetics), SatHb, PO2, etc.), as well as the various plots 904-908 generated in connection with the measurements. The target temperature (e.g., 37° C.) is used to set the temperature of the blood sample 132. The first stop condition or second stop condition are used to stop the measurements performed by apparatus (instrument) 162, as particularly described in reference to FIGS. 6 and 7. The increment of the capillary is used to set an offset of the capillary 130 from its previous position in order to take a further measurements associated with the method that does not use enzymatic deoxygenation. If the increment is not set or is set to zero (e.g., “0”), all measurements are taken at the meniscus 233 of the capillary 130 without offset associated with the method that uses enzymatic deoxygenation. The start and stop buttons allow the user to respectively start and stop the experiment, as described herein with reference to FIGS. 6 and 7.

The second set of parameters (right column) of the dashboard represents current measurements of the system 100. The second set of parameters can include the status of the apparatus (instrument) 162, the current or elapsed time, the current temperature of the blood sample 132, and current PO2 (mm Hg) and SatHb calculated during a current cycle.

The oxygen equilibrium curve (OEC) plot 904 is generated over a plurality of measurement cycles, and illustrates a respective plurality as a function SatHb(PO2).

The absorbance plot 908 illustrates a plurality of points that represent average absorbance spectrum measurements over a plurality of measurement cycles, and a curve (e.g., linear combination of Hb and OxyHb model curves) that fits these points.

Lastly, phosphorescence plot 908 illustrates average kinetics of phosphorescence decay of a phosphorescence measurement and an associated calculated exponential fit for a current measurement cycle.

In the foregoing, there have been described a system, apparatus, and method of testing a blood sample or a non-blood sample, providing for collection of absorbance spectrum and phosphorescence using a submillimeter-size diameter capillary and microliter amount of a blood sample, facilitating miniaturization of the apparatus, uniformity of temperature, and decrease in amounts of chemicals, and yet providing precise measurements for generation of the OEC. While the foregoing description addressed examples of testing a blood sample, the system, apparatus, and method are equally applicable to testing a non-blood sample capable of transporting (e.g., binding and releasing) oxygen, such as a liquid that includes a suspension of purified bovine or other red blood cells capable of transporting oxygen, as well as artificial blood that includes a special liquid, a suspension of vesicles, or other carriers capable of transporting oxygen.

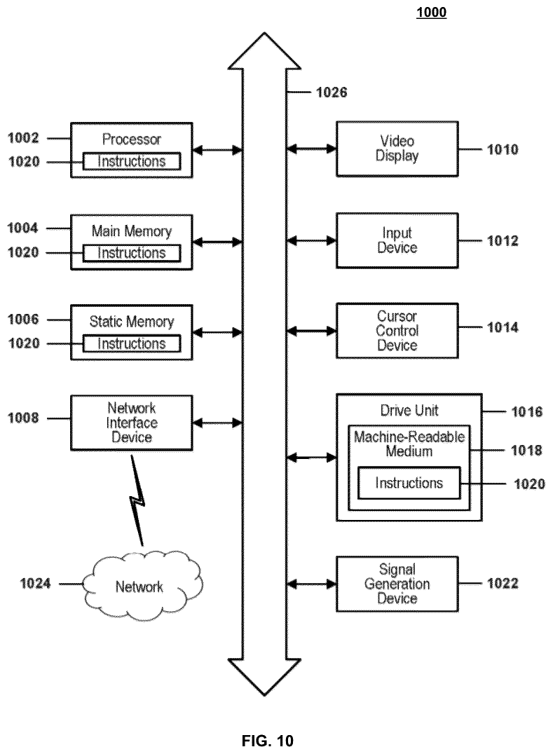

FIG. 10 illustrates a block diagram of an example general computer system 1000 capable of performing any methods or computer-based functions in accordance with FIGS. 1-9.

The computer system 1000 can include a set of instructions that can be executed to cause the computer system 1000 to perform any one or more of the methods or computer based functions disclosed herein in FIGS. 1-10. The computer system 1000, or any portion thereof, may operate as a standalone device or may be connected, e.g., using a network or other connection, to other computer systems or peripheral devices. For example, the computer system 1000 may be a computing device 164, or can even be implemented as the microcontroller 160.

The computer system 1000 may also be implemented as or incorporated into various devices, such as a personal computer (PC), a tablet PC, a personal digital assistant (PDA), a computing device or mobile device (e.g., smartphone), a palmtop computer, a laptop computer, a desktop computer, a communications device, a control system, a web appliance, or any other machine capable of executing a set of instructions (sequentially or otherwise) that specify actions to be taken by that machine. Further, while a single computer system 1000 is illustrated, the term “system” shall also be taken to include any collection of systems or sub-systems that individually or jointly execute a set, or multiple sets, of instructions to perform one or more computer functions.

As illustrated in FIG. 10, the computer system 1100 may include a processor 1002, e.g., a central processing unit (CPU), a graphics-processing unit (GPU), or both. Moreover, the computer system 1000 may include a main memory 1004 and a static memory 1006 that can communicate with each other via a bus 1026. As shown, the computer system 1000 may further include a video display unit 1010, such as a liquid crystal display (LCD), an organic light emitting diode (OLED), a flat panel display, a solid state display, or a cathode ray tube (CRT). Additionally, the computer system 1000 may include an input device 1012, such as a keyboard, and a cursor control device 1014, such as a mouse. The computer system 1000 can also include a disk drive (or solid state) unit 1016, a signal generation device 1022, such as a speaker or remote control, and a network interface device 1108.

In a particular embodiment or aspect, as depicted in FIG. 10, the disk drive (or solid state) unit 1016 may include a computer-readable medium 1018 in which one or more sets of instructions 1020, e.g., software, can be embedded. Further, the instructions 1020 may embody one or more of the methods or logic as described herein. In a particular embodiment or aspect, the instructions 1020 may reside completely, or at least partially, within the main memory 1004, the static memory 1006, and/or within the processor 1002 during execution by the computer system 1000. The main memory 1004 and the processor 1002 also may include computer-readable media.

In an alternative embodiment or aspect, dedicated hardware implementations, such as application specific integrated circuits, programmable logic arrays and other hardware devices, can be constructed to implement one or more of the methods described herein. Applications that may include the apparatus and systems of various embodiments or aspects can broadly include a variety of electronic and computer systems. One or more embodiments or aspects described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments or aspects, the methods described herein may be implemented by software programs tangibly embodied in a processor-readable medium and may be executed by a processor. Further, in an exemplary, non-limited embodiment or aspect, implementations can include distributed processing, component/object distributed processing, and parallel processing. Alternatively, virtual computer system processing can be constructed to implement one or more of the methods or functionality as described herein.

It is also contemplated that a computer-readable medium includes instructions 1020 or receives and executes instructions 1020 responsive to a propagated signal, so that a device connected to a network 1024 can communicate voice, video or data over the network 1024. Further, the instructions 1020 may be transmitted or received over the network 1024 via the network interface device 1008.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, example embodiment or aspect, the computer-readable medium can include a solid-state memory, such as a memory card or other package, which houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

In accordance with various embodiments or aspects, the methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that software that implements the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein, and other equivalents and successor media, in which the software implementations herein may be stored, are included herein.

Thus, a system, apparatus, and method directed to testing a blood sample or a non-blood sample capable of transporting oxygen in order to generate an oxygen equilibrium curve have been described. Although specific example embodiments or aspects have been described, it will be evident that various modifications and changes may be made to these embodiments or aspects without departing from the broader scope of the invention. Accordingly, the specification and drawings are to be regarded in an illustrative rather than a restrictive sense. The accompanying drawings that form a part hereof, show by way of illustration, and not of limitation, specific embodiments or aspects in which the subject matter may be practiced. The embodiments or aspects illustrated are described in sufficient detail to enable those skilled in the art to practice the teachings disclosed herein. Other embodiments or aspects may be utilized and derived therefrom, such that structural and logical substitutions and changes may be made without departing from the scope of this disclosure. This Detailed Description, therefore, is not to be taken in a limiting sense, and the scope of various embodiments or aspects is defined only by the appended claims, along with the full range of equivalents to which such claims are entitled.

Such embodiments or aspects of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments or aspects have been illustrated and described herein, it should be appreciated that any arrangement calculated to achieve the same purpose may be substituted for the specific embodiments or aspects shown. This disclosure is intended to cover any and all adaptations or variations of various embodiments or aspects. Combinations of the above embodiments or aspects, and other embodiments or aspects not specifically described herein, will be apparent to those of skill in the art upon reviewing the above description.

The Abstract is provided to comply with 37 CFR § 1.72(b) and will allow the reader to quickly ascertain the nature and gist of the technical disclosure. It is submitted with the understanding that it will not be used to interpret or limit the scope or meaning of the claims.

In the foregoing description of the embodiments or aspects, various features are grouped together in a single embodiment for the purpose of streamlining the disclosure. This method of disclosure is not to be interpreted as reflecting that the claimed embodiments or aspects have more features than are expressly recited in each claim. Rather, as the following claims reflect, inventive subject matter lies in less than all features of a single disclosed embodiment or aspect. Thus the following claims are hereby incorporated into the Detailed Description, with each claim standing on its own as a separate example embodiment or aspect. It is contemplated that various embodiments or aspects described herein can be combined or grouped in different combinations that are not expressly noted in the Detailed Description. Moreover, it is further contemplated that claims covering such different combinations can similarly stand on their own as separate example embodiments or aspects, which can be incorporated into the Detailed Description.

1. A system to test a sample capable of transporting oxygen and comprising at least a phosphorescence dye, the system comprising:
an illumination device configured to generate a first light pulse for a first time period and a plurality of second light pulses each for a second time period, the first light pulse and the second light pulses capable of illuminating the sample;
a first light collector configured to collect a plurality of absorbance spectrum measurements from the sample illuminated by the first light pulse;
a second light collector configured to collect a plurality of phosphorescence measurements emitted by the phosphorous dye of the sample illuminated by the plurality of second light pulses, each phosphorescence measurement being collected during a third time period after a corresponding one of the second light pulses;
a microcontroller configured to generate an absorbance spectrum average based on the plurality of absorbance spectrum measurements, and further configured to generate a phosphorescence average based on the plurality of phosphorescence measurements;
a computing device configured to calculate a partial pressure of oxygen (PO2) based on the phosphorescence average and calculate an oxygen saturation of hemoglobin (SatHb) based on the absorbance spectrum average, the computing device further configured to plot a point represented by SatHb, PO2 on an oxygen equilibrium curve.

2. The system of claim 1, wherein the illumination device comprises:
a first light source configured to generate the first light pulse; and
a second light source configured to generate the plurality of second light pulses.

3. The system of claim 2, wherein the illumination device comprises a light combiner configured to spatially combine the first light pulse of the first light source and the second pulses of the second light source along an optical axis.

4. The system of claim 3, wherein the light combiner is a beam splitter configured to allow the first light pulse to pass along the optical axis and further configured to reflect the second light pulse along the optical axis.

5. The system of claim 3, wherein the first light collector is positioned along the optical axis in order to collect the plurality of absorbance spectrum measurements, and the second light collector is positioned at an angle with respect to the optical axis in order to collect the plurality of phosphorescence measurements, wherein the angle is in a range of 20° to 120°.

6. The system of claim 5, wherein the angle is 60°.

7. The system of claim 2, wherein the first light pulse is in a range of 450 nm to 650 nm.

8. The system of claim 2, wherein the second light pulse is in a range of 620 nm to 635 nm.

9. The system of claim 1, wherein the first light collector comprises:
an optical aperture configured to receive a light transmitted through the sample illuminated by the first light pulse; and
a spectral detector configured to detect the plurality of absorbance spectrum measurements from the sample.

10. The system of claim 9, wherein the optical aperture is an optical fiber.

11. The system of claim 9, wherein the spectral detector is a spectrometer.

12. The system of claim 1, wherein the second light collector comprises a phosphorescence detector configured to collect the plurality of phosphorescence measurements emitted by the phosphorescence dye.

13. The system of claim 12, wherein the phosphorescence dye has an absorbance in a range 637 nm±10 nm, and an emission in a range 813±20 nm.

14. The system of claim 1, further comprising a heating sample stage configured to maintain the sample at a predetermined temperature.

15. The system of claim 1, further comprising a position sample stage configured to translate a capillary with the sample along a capillary axis and/or an optical axis.

16. The system of claim 15, wherein:
the capillary with the sample is configured to be translated along the capillary axis to an initial position at a predetermined distance from the meniscus of the sample with respect to the optical axis for a first cycle of measurement, and the capillary with the sample is further configured to be translated along the capillary axis by a predetermined distance from the initial position for a subsequent cycle of measurements; or
the capillary with the sample is configured to be translated along the capillary axis to an initial position of the meniscus of the sample with respect to the optical axis for measurements of a plurality of cycles, and a gas flow is configured to be directed toward the meniscus of the sample for the measurements of the plurality of cycles.

17. The system of claim 11, wherein the sample is a blood sample or a non-blood sample.

18. The system of claim 17, wherein the sample is the non-blood sample, the non-blood sample being a liquid that includes a suspension of purified bovine or other red blood cells capable of transporting oxygen, or artificial blood that includes a special liquid, a suspension of vesicles, or other carriers capable of transporting oxygen.

19. An apparatus to test a sample capable of transporting oxygen and comprising at least a phosphorescence dye, the apparatus comprising:
an illumination device configured to generate a first light pulse for a first time period and a plurality of second light pulses each for a second time period, the first light pulse and the second light pulses capable of illuminating the sample;
a first light collector configured to collect a plurality of absorbance spectrum measurements from the sample illuminated by the first light pulse;
a second light collector configured to collect a plurality of phosphorescence measurements emitted by the phosphorous dye of the sample illuminated by the plurality of second light pulses, each phosphorescence measurement being collected during a third time period after a corresponding one of the second light pulses; and
a microcontroller configured to generate an absorbance spectrum average based on the plurality of absorbance spectrum measurements, configured to generate a phosphorescence average based on the plurality of phosphorescence measurements, and further configured to transmit the absorbance spectrum average and the phosphorescence average to a computing device to calculate a partial pressure of oxygen (PO2) based on the phosphorescence average and an oxygen saturation of hemoglobin (SatHb) based on the absorbance spectrum average, and to plot a point represented by SatHb, PO2 on an oxygen equilibrium curve.

20. The apparatus of claim 19, wherein the illumination device comprises:
a first light source configured to generate the first light pulse; and
a second light source configured to generate the plurality of second light pulses.

21. The apparatus of claim 20, wherein the illumination device comprises a light combiner configured to spatially combine the first light pulse of the first light source and the second pulses of the second light source along an optical axis.

22. The apparatus of claim 21, wherein the light combiner is a beam splitter configured to allow the first light pulse to pass along the optical axis and further configured to reflect the second light pulse along the optical axis.

23. The apparatus of claim 21, wherein the first light collector is positioned along the optical axis in order to collect the plurality of absorbance spectrum measurements, and the second light collector is positioned at an angle with respect to the optical axis in order to collect the plurality of phosphorescence measurements, wherein the angle is in a range of 20° to 120°.

24. The apparatus of claim 23, wherein the angle is 60°.

25. The apparatus of claim 20, wherein the first light pulse is in a range of 450 nm to 650 nm.

26. The apparatus of claim 20, wherein the second light pulse is in a range of 620 nm to 635 nm.

27. The apparatus of claim 19, wherein the first light collector comprises:
an optical aperture configured to receive a light transmitted through the sample illuminated by the first light pulse; and
a spectral detector configured to detect the plurality of absorbance spectrum measurements from the sample.

28. The apparatus of claim 27, wherein the optical aperture is an optical fiber.

29. The apparatus of claim 27, wherein the spectral detector is a spectrometer.

30. The apparatus of claim 19, wherein the second light collector comprises a phosphorescence detector configured to collect the plurality of phosphorescence measurements emitted by the phosphorous dye.

31. The apparatus of claim 30, wherein the phosphorescence dye has an absorbance in a range 637 nm±10 nm, and an emission in a range 813±20 nm.

32. The apparatus of claim 19, further comprising a heating sample stage configured to maintain the sample at a predetermined temperature.

33. The apparatus of claim 19, further comprising a position sample stage configured to translate a capillary with the sample along a capillary axis and/or an optical axis.

34. The apparatus of claim 33, wherein:
the capillary with the sample is configured to be translated along the capillary axis to an initial position at a predetermined distance from the meniscus of the sample with respect to the optical axis for a first cycle of measurements; and the capillary with the sample is further configured to be translated along the capillary axis by a predetermined distance from the initial position for a subsequent cycle of measurements; or
the capillary with the sample is configured to be translated along the capillary axis to an initial position of the meniscus of the sample with respect to the optical axis for measurements of a plurality of cycles, and a gas flow is configured to be directed toward the meniscus of the sample for the measurements of the plurality of cycles.

35. The apparatus of claim 19, wherein the sample is a blood sample or a non-blood sample.

36. The apparatus of claim 35, wherein the sample is the non-blood sample, the non-blood sample being a liquid that includes a suspension of purified bovine or other red blood cells capable of transporting oxygen, or artificial blood that includes a special liquid, a suspension of vesicles, or other carriers capable of transporting oxygen.

37. A method of testing a sample capable of transporting oxygen and comprising at least a phosphorescence dye, the method comprising:
generating via an illumination device a first light pulse for a first time period and a plurality of second light pulses each for a second time period, the first light pulse and the second light pulses capable of illuminating the sample;
collecting via a first light collector a plurality of absorbance spectrum measurements from the sample illuminated by the first light pulse;
collecting via a second light collector a plurality of phosphorescence measurements emitted by the phosphorous dye of the sample illuminated by the plurality of second light pulses, each phosphorescence measurement being collected during a third time period after a corresponding one of the second light pulses;
generating via a controller an absorbance spectrum average based on the plurality of absorbance spectrum measurements, and further generating a phosphorescence average based on the plurality of phosphorescence measurements;
calculating via a computing device a partial pressure of oxygen (PO2) based on the phosphorescence average and calculating an oxygen saturation of hemoglobin (SatHb) based on the absorbance spectrum average; and
plotting via the computing device a point represented by SatHb, PO2 on an oxygen equilibrium curve.

38. The method of claim 37, wherein the method further comprises:
generating the first light pulse via a first light source of the illumination device; and
generating the plurality of second light pulses via a second light source of the illumination device.

39. The method of claim 38, wherein the method further comprises spatially combining the first light pulse of the first light source and the second light pulses of the second light source along an optical axis.

40. The method of claim 39, wherein the method further comprises passing the first light pulse along the optical axis and reflecting the second light pulse along the optical axis via a beam splitter.

41. The method of claim 39, wherein the method further comprises positioning the first light collector along the optical axis in order to collect the plurality of absorbance spectrum measurements, and positioning the second light collector at an angle with respect to the optical axis in order to collect the plurality of phosphorescence measurements, wherein the angle is in a range of 20° to 120°.

42. The method of claim 41, wherein the angle is 60°.

43. The method of claim 38, wherein the first light pulse is in a range of 450 nm to 650 nm.

44. The method of claim 38, wherein the second light pulse is in a range of 620 nm to 635 nm.

45. The method of claim 37, wherein the method further comprises:
receiving a light transmitted through the sample illuminated by the first light pulse via an optical aperture of the first light collector; and
detecting the plurality of absorbance spectrum measurements from the sample via a spectral detector of the first light collector.

46. The method of claim 45, wherein the optical aperture is an optical fiber.

47. The method of claim 45, wherein the spectral detector is a spectrometer.

48. The method of claim 37, wherein the method further comprises collecting the plurality of phosphorescence measurements emitted by the phosphorous dye via a phosphorescence detector of the second light collector.

49. The method of claim 48, wherein the phosphorescence dye has an absorbance in a range 637 nm±10 nm, and an emission in a range 813±20 nm.

50. The method of claim 37, wherein the method further comprises maintaining the sample at a predetermined temperature via a heating sample stage.

51. The method of claim 37, further comprising translating a capillary with the sample along a capillary axis and/or an optical axis via a position sample stage.

52. The method of claim 51, wherein the method further comprises:
translating via the position sample stage the capillary with the sample along the capillary axis to an initial position at a predetermined distance from the meniscus of the sample with respect to the optical axis for a first cycle of measurements; and translating via the position sample stage the capillary with the sample along the capillary axis by a predetermined distance from the initial position for a subsequent cycle of measurements; or
translating via the position sample stage the capillary with the sample along the capillary axis to an initial position of the meniscus of the sample with respect to the optical axis for measurements of a plurality of cycles, and flowing a gas via a tube directed toward the meniscus of the sample for the measurements of the plurality of cycles.

53. The method of claim 37, wherein the sample is a blood sample or a non-blood sample.

54. The method according to claim 53, wherein the sample is the non-blood sample, the non-blood sample being a liquid that includes a suspension of purified bovine or other red blood cells capable of transporting oxygen, or artificial blood that includes a special liquid, a suspension of vesicles, or other carriers capable of transporting oxygen.
